(19) United States
(12) Patent Application Publication
(10) Pub. No.: US 2011/0002843 A1
(43) Pub. Date: Jan. 6, 2011

(54) METHOD OF DETECTING MYOCARDIAL DYSFUNCTION IN PATIENTS HAVING A HISTORY OF ASTHMA OR BRONCHOSPASM

(76) Inventor: Richard J. Barrett, Cary, NC (US)

Correspondence Address: KING PHARMACEUTICALS, INC., 400 CROSSING BOULEVARD, BRIDGEWATER, NJ, 08807, US

(21) Appl. No.: 12/880,296

(22) Filed: Sep. 13, 2010

Related U.S. Application Data
(63) Continuation of application No. 11/332,120, filed on Jan. 12, 2006.
(60) Provisional application No. 60/643,481, filed on Jan. 12, 2005.

(51) Int. Cl. A61K 51/00; A61K 49/00; A61P 43/00

(52) U.S. Cl. 424/1.11; 424/9.1

(57) ABSTRACT

This invention is directed to myocardial imaging of human patients having a history of asthma or bronchospasm. In particular, the present invention uses binodenoson as a pharmacological stressor in conjunction with any one of several noninvasive and invasive diagnostic procedures available. For example, intravenous administration may be used in conjunction with a radiopharmaceutical agent and myocardial perfusion imaging to assess the severity of myocardial ischemia.

This application is a continuation of U.S. application Ser. No. 11/332,120, filed Jan. 12, 2006, which claims the benefit of U.S. Provisional Application No. 60/643,481, filed Jan. 12, 2005, both of which are hereby incorporated by reference in their entirety.

### FIELD OF THE INVENTION

The invention relates to methods of detecting and/or diagnosing myocardial dysfunction in human patients having a history of asthma or bronchospasm. In particular, the present invention uses binodenoson or other selective adenosine A_{2A} agonists as pharmacological stressors in conjunction with any one of several noninvasive and invasive diagnostic procedures available.

### BACKGROUND OF THE INVENTION

Adenosine has been known since the early 1920's to have potent vasodilator activity. It is a local hormone released from most tissues in the body during stress, especially hypoxic and ischemic stress (Olsson et al., Physiological Reviews, 70(3), 761-845, 1990). As such, adenosine and adenosine-releasing agents are now commonly used to simulate the stress condition for diagnostic purposes (A. N. Clark and G. A. Beller, “The present role of nuclear cardiology in clinical practice”, Quarterly Journal of Nuclear Medicine and Molecular Imaging, 49, 43-58, 2005).

Myocardial perfusion imaging is currently the most common approach in the use of stress-simulating agents (pharmacological stressors) as a means of imaging the coronary vessels to obtain a diagnosis of coronary artery disease. This is effected by injection of the pharmacological stressor such as adenosine at a dose of about 1 mg/kg body weight, followed by injection of an imaging agent, e.g., a radionuclide and imaging of the heart to detect the extent of any coronary circulation disorders.

The mechanism underlying myocardial perfusion imaging is as follows: adenosine acting on coronary adenosine receptors causes relaxation of the coronary arterioles, thereby increasing blood flow throughout the heart. This effect is short-lasting and at a dose of 1 mg/kg, adenosine does not dilate other peripheral blood vessels to produce substantial systemic hypotension. Diseased or otherwise blocked coronary vessels will not further dilate in response to adenosine and the subsequent flow of an imaging agent through the heart will be less in these regions of hypoperfusion relative to other more normal areas of the heart. The resulting image allows the diagnostician to quantify the amount and severity of the coronary perfusion defect. This analysis is of paramount importance in selecting any further course of therapy and intervention by the physician (U.S. Pat. No. 5,070,877 and No. 4,824,660).

The use of adenosine and like-acting analogs is associated with certain side effects. Adenosine acts on at least three subclasses of adenosine receptors; A₁, A₂ and A₃. The A₂ receptor subtype is found in blood vessels and is further divided into A_{2A} and A_{2B} receptor subtypes (Martin et al., Journal of Pharmacology and Experimental Therapeutics, 265(1), 248-253, 1993). While not being bound by any specific theory, it is believed that the A_{2A} receptor is responsible for mediating coronary vasodilation and providing the desired action of adenosine in the diagnostic procedure. The A₁ receptor subtype, when activated by adenosine, among other actions, slows the frequency and conduction velocity of the electrical activity that initiates the heartbeat. Sometimes adenosine, particularly at doses near 1 mg/kg, even blocks (stops) the heartbeat during this diagnostic procedure which is a highly undesirable action.

Another side effect associated with the administration of adenosine is bronchoconstriction in asthmatic patients. Bronchoconstriction has been associated with activation of the adenosine A₃ receptors on mast cells. (Linden J., Trends. Pharmacol. Sci., 15, 298-306, 1994). Furthermore, adenosine has been described as an asthma provoking agent in U.S. Pat. No. 6,248,723. Thus, the side effects of adenosine and adenosine releasing agents result substantially from non-selective stimulation of the various adenosine receptor subtypes.

Due to the side effects associated with administration of adenosine and, in particular, bronchoconstriction, patients afflicted with a history of asthma or bronchospasm have been excluded from methods of myocardial imaging using adenosine, dypyrimidamole and adenosine analogs as pharmacological stressors. Included in the class of excluded patients are patients having symptoms such as wheezing or a history of severe bronchospasm. These symptoms are often manifested in patients suffering from asthma or chronic obstructive pulmonary disorder (COPD).

Asthma, in particular, is a significant disease of the lung that affects nearly 12 million Americans. Asthma is typically characterized by periodic airflow limitation and/or hyper responsiveness to various stimuli that results in excessive airways narrowing. Other characteristics can include inflammation of airways, eosinophilia and airway fibrosis.

Asthma prevalence (i.e., both incidence and duration) is increasing. The current prevalence approaches 10% of the population and has increased 25% in the last 20 years. Of more concern, however, is the rise in the death rate. When coupled with increases in emergency room visits and hospitalizations, recent data suggests that asthma severity is rising. While most cases of asthma are easily controlled, for those with more severe disease, the costs, the side effects and all too often, the ineffectiveness of the treatment, present serious problems.

COPD is characterized by chronic inflammation of the small airways (<2 mm) which unavoidably results in tissue reconstruction and irreparable narrowing (obstruction) of this portion of the airways. Patients suffering from COPD typically show a decreased maximal expiratory flow and a slow forced emptying of the lungs. COPD is often associated with chronic bronchitis and emphysema.

Besides adenosine, other common pharmacological stressors for use in myocardial imaging include dipyridamole and dobutamine. Dipyridamole inhibits the uptake of adenosine into cells which enhances the extracellular effects of endogeneous adenosine. Similar to adenosine, dipyridamole is excluded for use as the pharmacological stressor with asthma patients and patients with a history of bronchospasm.

Dobutamine may be used as the pharmacological stressors in myocardial imaging of patients suffering from a pulmonary disorder with a history of asthma or bronchospasm. However, dobutamine has certain disadvantages as compared with adenosine. For instance, dobutamine side effects are frequently seen in patients. These side effects include ventricular arrythmias (or ectopy), chest pain, palpitations, headache, flushing and dyspnea. Side effects may also include atrial fibrillation or supraventricular tachycardia. Furthermore, angina with ST segment depression is reported to occur in a number of patients with coronary artery disease.

U.S. Pat. No. 5,477,857 (“the '857 patent”) to McAfee et al. claims myocardial imaging use of 2-cyclohexylmethylhydrazinoadenosine. Although the '857 patent discloses that other hydrazinoadenosine compounds may be used, only the single compound method of use is claimed. The '857 patent also claims the method of myocardial imaging in mammals. No particular human usage is exemplified or disclosed.

Martin et al. generally discloses the pharmacological properties of 2-cyclohexylmethylidenehydrazinoadenosine (binodenoson) as compared with adenosine. (Drug Development Research, 40, 313-324, 1997). Among other things, Martin et al. compared the effect of certain doses of binodenoson on coronary blood flow with adenosine in anaesthetized and conscious dogs. Based on the doses that were reported to increase coronary vasodilation in dogs, similar doses were administered in an allergic sheep model of asthma in order to measure the effect on lung resistance. The following results were observed: binodenoson, unlike adenosine, did not increase lung resistance in sheep, however, sheep administered binodenoson experienced a significant increase in respiratory rate. Thus, in the sheep model, the authors did not report a dose of binodenoson that would avoid adverse effects (AEs), such as, an increased respiratory rate.

In sum, there remains a need for administering dosages of binodenoson that safely achieve coronary vasodilation in human patients with a history of asthma or bronchospasm without simultaneous bronchoconstriction to enable a broader patient population to undergo a myocardial imaging method. In addition, since coronary hyperemic responses to binodenoson alter the coronary blood flow in a vulnerable patient population, i.e., those who might suffer from coronary blood flow disorders, the hyperemic effects achieved by such methods for administering and dosages of binodenoson should be readily reversible.

### SUMMARY OF THE INVENTION

In one aspect, the invention relates to a method of diagnosing myocardial dysfunction in a human patient having a history of asthma or bronchospasm. The method includes the steps of:

(a) administering by an intravenous route to the human patient from about 0.1 to about 10 μg/kg of binodenoson to provide coronary artery dilation; and

(b) detecting myocardial dysfunction in the human patient.

In some embodiments of the method, binodenoson is administered as a bolus dose to said human patient. For example, in a specific embodiment, from about 0.5 to about 2.5 μg/kg of the binodenoson is administered to said human patient.

In other embodiments of the method, binodenoson is administered by infusion to said human patient. For example, in a specific embodiment, from about 0.3 to about 2.0 μg/kg/min of the binodenoson is administered to said human patient.

In specific embodiments of the method, the myocardial dysfunction is coronary artery disease, ventricular dysfunction, differences in blood flow through disease free coronary vessels and stenotic vessels, or a combination thereof.

In some embodiments of the method, step (b) comprises a noninvasive myocardial imaging procedure. For instance, in a specific embodiment, the noninvasive imaging procedure includes administration of an imaging agent.

In another aspect, the invention relates to a method of detecting and/or diagnosing coronary artery disease in a human patient having a history of asthma or bronchospasm. The method of detecting coronary artery disease includes the steps of:

(a) administering by an intravenous route to the human patient from about 0.1 to about 10 μg/kg of binodenoson to provide coronary artery dilation;

(b) administering an imaging agent to the human patient; and

(c) performing myocardial perfusion imaging on the human patient to detect coronary artery disease.

In another aspect, the invention relates to a method of detecting and/or diagnosing ventricular dysfunction caused by coronary artery disease in a human patient having a history of asthma or bronchospasm. The method of detecting ventricular dysfunction includes the steps of:

(a) administering by an intravenous route to the human patient from about 0.1 to about 10 μg/kg of binodenoson to provide coronary artery dilation; and

(b) performing a ventricular function imaging technique on the human patient to detect ventricular dysfunction.

In yet another aspect, the invention relates to a method of detecting and/or diagnosing perfusion abnormalities in a human patient having a history of asthma or bronchospasm. The method of detecting perfusion abnormalities includes the steps of:

(a) administering by an intravenous route to the human patient from about 0.1 to about 10 μg/kg of binodenoson to provide coronary artery dilation; and

(b) detecting perfusion abnormalities in the human patient.

In certain embodiments of the method of detecting perfusion abnormalities, step (b) comprises measuring coronary blood flow velocity on the human patient to assess the vasodilatory capacity of diseased coronary vessels as compared with disease free coronary vessels. In other embodiments of the method, step (b) comprises assessing the vasodilatory capacity (reserve capacity) of diseased coronary vessels as compared with disease-free coronary vessels.

In another aspect, the invention relates to a method of detecting the presence and assessing the severity of coronary artery disease in a human patient having a history of asthma or bronchospasm. The method includes the steps of:

(a) administering by an intravenous route to the human patient from about 0.1 to about 10 μg/kg of binodenoson to provide coronary artery dilation;

(b) administering a radiopharmaceutical agent to the human patient; and

(c) performing scintigraphy on the human patient to detect the coronary artery disease.

In still another aspect, the invention relates to a method of detecting the presence and assessing the severity of ventricular dysfunction in a human patient having a history of asthma or bronchospasm. The method includes the steps of:

(a) administering by an intravenous route to the human patient from about 0.1 to about 10 μg/kg of binodenoson to provide coronary artery dilation; and

(b) performing echocardiography on the human patient to detect ventricular dysfunction.

In another aspect, the invention relates to a kit comprising, a first container containing a unit dosage of binodenoson and a second container containing an imaging agent, an adenosine antagonist or a β-2 agonist.

### BRIEF DESCRIPTION OF THE DRAWINGS

FIG. 1 shows the mean forced expiratory volume in 1 second (FEV₁) over time in placebo and binodenoson (1.5 μg/kg) treated human patients with mild, intermittent asthma.

FIG. 2 is a graph showing maximal coronary hyperemic responses to 3-minute infusions of 0.9, 1.5 and 3 μg/kg; and to bolus doses (over 30 seconds) of 1.5 and 3 μg/kg binodenoson in 25-28 human (non-asthmatic) patients. Responses expressed as mean±standard deviation (SD) percent of the coronary blood flow velocity reserve (CBFVR).

FIG. 3 is a graph showing time-course of mean CBFV responses, expressed as percent of CBFVR, of 5 binodenoson doses in human (non-asthmatic) patients.

FIG. 4 is a graph showing the effect over time of binodenoson, a 1.5 μg/kg bolus dose, on coronary blood flow velocity (CBFV), coronary vascular resistance (CVR), systolic blood pressure (SBP), diastolic blood pressure (DBP) and heart rate (HR) in human (non-asthmatic) patients.

FIG. 5 is a graph showing the mean (±SD) concentrations of binodenoson after administration of 3 μg/kg over a period of 10 minutes in non-asthmatic human patients.

FIG. 6 is a graph showing the relationship between binodenoson AUC₀₋ₜ and total dose (in micrograms) in non-asthmatic human patients.

FIG. 7 is a graph showing the relationship between binodenoson systemic clearance (CL) and body weight in non-asthmatic human patients.

FIG. 8 is a histogram showing the number of adverse effects per subject associated with doses of binodenoson in non-asthmatic human patients.

FIG. 9A shows the mean (SD) maximal changes in heart rate at different doses of binodenoson in non-asthmatic human patients.

FIG. 9B shows the mean (SD) maximal changes in systolic and diastolic pressure in non-asthmatic human patients.

FIG. 10 is a graph showing the simulated binodenoson concentrations in the systemic circulation after administration of 1.5 μg/kg over periods of 10 minutes, 3 minutes and 30 seconds.

### DETAILED DESCRIPTION OF THE INVENTION

Provided are methods of detecting myocardial dysfunction in human patients with pulmonary disorders having a reactive airway component, e.g., such as patients with asthma or COPD. Among other things, the inventive methods enable a broader patient population to benefit from known myocardial dysfunction diagnostic procedures that rely on increasing coronary blow flow by administration of pharmacological stressors. Since the inventive methods use selective A_{2A} agonists such as binodenoson to provide coronary dilation and thereby increase the coronary blood flow, the methods substantially reduce, or eliminate the undesired side effects that accompany use of other pharmacological stressors such as adenosine, dipyrimadole or dobutamine. The improvements are especially important in patients suffering from a pulmonary disorder with a reactive airways component, where fewer pharmacological stressors can be safely used as compared with patients that are free of pulmonary disorders.

In one embodiment, for instance, the inventive methods are useful in detecting myocardial dysfunction in patients having a history of asthma or bronchospasm. In some embodiments, such patients may be identified by referral to the patient's medical history to detect a history of a pulmonary disorder with a reactive airways component, e.g., asthma or bronchospasm. Alternatively, patients having mild, asthma can be identified at a screening interview or consultation by confirming reversal of bronchoconstriction following administration of albuterol. In another embodiment, patients with asthma can be identified by a positive challenge to a metacholine challenge test.

Pharmacological Stressors: Suitable compounds for use as pharmacological stressors in the present invention are potent and selective agonists of the adenosine A_{2A} receptor. In a specific embodiment, the pharmacological stressors act as agonists of the adenosine A_{2A} receptor with a coronary vasodilation EC₅₀ of coronary vasodilation less than 2.5 nM and a selectivity index quotient as compared to the adenosine A₁ receptor of at least 10,000 and a selectivity quotient as compared to the adenosine A_{2B} receptor of at least 10,000. In preferred embodiments, the compounds have been further tested for side effects deleterious to human patients suffering from a disorder with a reactive airways component, such as patients who suffer from asthma or bronchospasm.

Compounds selective as agonists for human A_{2A} receptors are disclosed in U.S. Pat. No. 5,278,150 to Olsson et al. (“the '150 patent”), which is hereby incorporated by reference in its entirety. The compounds described in the '150 patent, in general, are 2-substituted hydrazino adenosines. Selectivity and potency of the compounds in the '150 patent vary greatly. The patent discloses only A₁/A_{2A} selectivity and potency for such compounds. Testing for suitability for use with the present invention typically requires in addition, measuring A_{2B}/A_{2A} selectivity and determining if the compounds have acceptable levels of side-effects for human patients having history of asthma or bronchospasm.

Additional adenosine compounds potent for A_{2A} receptors are disclosed in U.S. Pat. No. 6,326,359 to Monaghan et al (“the '359 patent”), which is hereby incorporated by reference in its entirety. While it is believed that some of the compounds in the '359 patent may be suitable, data for potency, A₁/A_{2A} and A_{2B}/A_{2A} selectivity is not presently available. Use of the present invention with compounds disclosed in the '359 patent therefore requires such testing as well as testing for side effects in human patients having history of asthma or bronchospasm.

In specific embodiments, the pharmacological stressors are selected from:
2-{2-[(Cyclohexyl)methylene]hydrazino}adenosine (binodenoson),
2-{2-[(Cyclohex-3-enyl)methylene]hydrazino}adenosine,
2-[2-(4-methylpentylidene)hydrazino]adenosine,
2-[2-(3-ethylheptylidene)hydrazino]adenosine,
2-[2-(hexylidene)hydrazino]adenosine,
2-[2-(4-Methoxybenzylidene)hydrazino]adenosine,
2-[2-(4-propylheptylidene)hydrazino]adenosine,
2-[2-(3-Propylbenzylidine)hydrazino]adenosine,
2-[2-(Benzylidene)hydrazino]adenosine,
2-[2-(4-Fluorobenzylidene)hydrazino]adenosine,
2-[2-(4-Methylbenzylidine)hydrazino]adenosine,
2-[2-(3-Methylbenzylidine)hydrazino]adenosine, or
2-[2-(4-Chlorobenzylidene)hydrazino]adenosine.

Compounds can be assessed for their suitability as pharmacological stressors by known methods to determine the potency and selectivity of compounds for the adenosine A_{2A} receptor. In one embodiment, a Langendorff guinea pig heart preparation paced at 260 beats/min, via the left atrium served for assays of A₁ adenosine receptor and A_{2A} adenosine receptor agonist activity (J. Med. Chem., 34, 1334-1339, 1991; and U.S. Pat. No. 5,278,150). The perfusion buffer consisted of 120 mM NaCl, 27 mM NaHCO₃, 3.7 mM KCl, 1.3 mM KH₂PO₄, 0.64 mM MgSO₄, 1.3 mM CaCl₂, 2 mM pyruvate and 5 mM glucose. The buffer was saturated with 95% O₂/5% CO₂, equilibrated at 37° C. in a heat exchanger and delivered at a pressure equivalent to 55 mmHg Continuous drainage of the left ventricle by means of a catheter inserted across the mitral valve insured that this cardiac chamber did no external work. An electrode in the right ventricle monitored the electrocardiogram. Timed collections of cardiac effluent in a graduated cylinder during the steady-state phase of the flow responses to compound administration measured total coronary flow, which was also monitored by an in-line electromagnetic flowmeter in the aortic perfusion cannula. The quotient of the ratio of compound infusion (mol/min) divided by coronary flow rate (L/min) equals agonist concentration in the perfusate. The rate of agonist infusion was increased stepwise at intervals of 3-4 minutes until the appearance of second degree heart block (Wenckebach point). The EC₅₀ of prolongation of the stimulus-QRS interval (EC₅₀-SQPR), the concentration of compound needed to prolong the interval by 50% of the maximum response, reflects activity at the adenosine A₁ receptor. Logit transformation of the coronary flow data and solution of the regression of logit (coronary flow) on log [compound] for logit=0 yielded an estimate of EC₅₀ of coronary vasodilation (EC₅₀-CF), an index of A₂ adenosine receptor activity. The quotient of the EC₅₀ of stimulus-QRS prolongation divided by the EC₅₀ of coronary vasodilation provided an index of selectivity. Values of the index >1 indicate selectivity for the A₂ adenosine receptor.

Certain highly selective potent agonists of the adenosine A_{2A} receptor have been disclosed, for instance, in U.S. Pat. No. 5,278,150 (“the 150 patent”). The '150 patent describes EC₅₀-SQPR and EC₅₀-CF data obtained for the following compounds in the Langendorff guinea pig heart preparation as described above. The results are shown below in Table 1. The A₁/A_{2A} selectivity in Table 1 was calculated as the quotient of the EC₅₀ of stimulus-QRS prolongation divided by the EC₅₀ of coronary vasodilation.

TABLE 1
Adenosine Receptor Binding and Selectivity

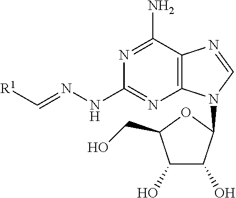

| | | EC₅₀—SQPR (A₁) | EC₅₀—CF (A_{2A}) | A₁/A_{2A} |
|---|---|---|---|---|
| Compound | R¹ | (nM) | (nM) | Selectivity |
| Binodenoson | Cyclohexyl | 3,550 | 0.26 | 13,800 |
| B | 3-Cyclo-hexenyl | 13,800 | 0.32 | 42,700 |
| C | 3-Me-1-Bu | 20,900 | 0.47 | 44,700 |
| D | 2-C Hexylethyl | 9,770 | 0.69 | 14,100 |
| E | 1-Pent | 38,900 | 1.02 | 38,000 |
| F | 4-MeO Ph | 22,900 | 1.74 | 13,200 |
| G | 3-C Hexylpropyl | 66,100 | 1.78 | 37,200 |
| H | 3-Ph Propyl | 66,100 | 1.95 | 33,900 |
| I | Ph | 83,200 | 2.29 | 36,300 |
| J | 4-F Ph | 12,600 | 2.45 | 5,100 |
| K | 4-Me Ph | 39,800 | 3.24 | 12,300 |
| L | 3-Me Ph | 17,000 | 4.40 | 3,800 |
| M | 4-Cl Ph | 14,100 | 4.47 | 3,200 |
| Compare A | Adenosine | 3,400 | 20.4 | 170 |
| Compare B | 2-Aminoadenosine | 11,200 | 220 | 50 |
| Compare C | 2-Hydrazinoadenosine | 19,900 | 80 | 250 |

As can be seen in Table 1, many 2-substituted hydrazino adenosine compounds show high affinity at the adenosine A₂ receptor with very good selectivity against the A₁ receptor. Most preferred are those compounds showing high A₂ potency (EC₅₀-CF<2.5) and high selectivity (selectivity>10,000).

TABLE 2
Identification of Compounds in Table 1

| Compound | Name |
|---|---|
| Binodenoson | 2-{2-[(Cyclohexyl)methylene]hydrazino}adenosine |
| B | 2-{2-[(Cyclohex-3-enyl)methylene]hydrazino}adenosine |
| C | 2-[2-(4-methylpentylidene)hydrazino]adenosine |
| D | 2-[2-(3-ethylheptylidene)hydrazino]adenosine |
| E | 2-[2-(hexylidene)hydrazino]adenosine |
| F | 2-[2-(4-Methoxybenzylidene)hydrazino]adenosine |
| G | 2-[2-(4-propylheptylidene)hydrazino]adenosine |
| H | 2-[2-(3-Propylbenzylidine)hydrazino]adenosine |
| I | 2-[2-(Benzylidene) hydrazino]adenosine |
| J | 2-[2-(4-Fluorobenzylidene) hydrazino]adenosine |
| K | 2-[2-(4-Methylbenzylidine) hydrazino]adenosine |
| L | 2-[2-(3-Methylbenzylidine)hydrazino]adenosine |
| M | 2-[2-(4-Chlorobenzylidene)hydrazino]adenosine |
| Compare A | Adenosine |
| Compare B | 2-Amino-adenosine |
| Compare C | 2-Hydrazino-adenosine |

It is further preferable to use compounds that are selective for the A_{2A} receptor over the A_{2B} receptor. Additional methods are known in the art for performing bioassays and are useful to identify selectivity to the A_{2B} receptor as well as confirming selectivity and potency in vivo. Such bioassays are typically performed prior to animal and human trials. Table 3 shows the results of prepared guinea pig assays for binodenoson, performed prior to human trials.

As is seen in Table 3, binodenoson is a potent A_{2A} agonist and is confirmed to be very selective as against adenosine A₁ and A_{2B} receptors. It is reasonable that additional compounds identified in Table 1 have corresponding results and are also suitable for use in the present invention.

TABLE 3
Bioassay Testing for Binodenoson

| | Adenosine | |
|---|---|---|
| Assay | Receptor | EC₅₀ (nM) |
| G.P. Right Atrium (Negative Inotropy) | A₁ | 21,000 |
| G.P. Left Atrium(Negative Inotropy) | A₁ | 38,900 |
| G.P. Right Atrium (Negative Chronotropy) | A₁ | 39,800 |
| G.P. Langendorf Heart (Negative Dromotrophy) | A₁ | 3,500 |
| G.P. Langendorf Heart (Coronary Dilation) | A_{2A} | 0.26 |
| G.P. Aortic Ring (Relaxation) | A_{2B} | 44,700 |

In a specific embodiment of the invention, the adenosine A_{2A} receptor agonist is binodenoson. Among other things, the administration of the selective A_{2A} agonist, binodenoson, achieves a useful level of coronary vasodilation without the need to subject the human patient to physical exercise. This property of binodenoson allows patients who are unable to exercise to be assessed by the detection methods described below. Therefore, in preferred embodiments of the methods of the invention, the patients need only be administered binodenoson to induce a level of coronary vasodilation to facilitate the detection procedures.

In alternative embodiments, the methods of the invention can be practiced wherein the human patient is subjected to physical exercise in an amount sufficient to contribute to the coronary artery dilation already induced by binodenoson. For example, the patient may walk or run on a treadmill prior to or simultaneously with the technique used to detect the presence and assess the severity of the myocardial dysfunction. In embodiments that combine physical exercise with the administration of binodenoson, lower doses of binodenoson may be administered.

Methods of Detecting Myocardial Dysfunction: In certain embodiments, the invention relates to a method of diagnosing myocardial dysfunction in a human patient having a history of asthma or bronchospasm. By way of embodiment, the invention is described using the adenosine A_{2A} receptor agonist, binodenoson. However, the skilled artisan will recognize that other selective adenosine A_{2A} receptor agonists such as those described above may be utilized in the inventive method after assessment of its selectivity as described in the preceding section and safety as described in Examples 1 and 3.

The method includes the steps of:

(a) administering by an intravenous route to the human patient from about 0.1 to about 10 μg/kg of binodenoson to provide coronary artery dilation; and

(b) detecting myocardial dysfunction in the human patient.

Detecting myocardial dysfunction can include detecting the presence of myocardial dysfunction in the human patient, the location of the myocardial dysfunction in the patient's heart, assessing the severity of the myocardial dysfunction in the human patient, or a combination thereof. The myocardial dysfunction may be, but is not limited to, coronary artery disease (e.g., stenosis of the coronary vessels), coronary wall abnormalities, ventricular dysfunction, valvular or congenital disease and cardiomyopathy, microvascular disease and myocardial viability.

Detection procedures which use binodenoson as a pharmacological stressor may be either noninvasive or invasive detecting procedures. Noninvasive detection procedures include those that image the myocardium or myocardial infarcts (myocardial perfusion imaging and myocardial infarct imaging). Furthermore, noninvasive detection procedures include those that permit an assessment of ventricular function and wall motion.

Imaging agents are often administered in noninvasive detection procedures. Typically, the imaging agents are injected into the patient after injection of the pharmacological stressor and then the clinician detects, records and analyzes the image (using, for example, a rotating gamma scintillation analyzer). Imaging agents include, but are not limited to, radiopharmaceuticals (such as for single photon emission computed tomography, positron emission tomography or computed tomography procedures), magnetic resonance imaging agents and microbubbles (such as for myocardial contrast echocardiography). Radiopharmaceuticals may be used in imaging procedures and include, but are not limited, to thallium-201, rubidium-82, technetium-99m, derivatives of technetium-99m, nitrogen-13, rubidium-82, iodine-123 and oxygen-15.

In some embodiments of the invention, the myocardial dysfunction is detected by myocardial perfusion imaging. The imaging can be performed by scintigraphy, single photon emission computed tomography (SPECT), positron emission tomography (PET), nuclear magnetic resonance (NMR) imaging, perfusion contrast echocardiography, digital subtraction angiography (DSA) and ultra fast X-ray computed tomography (CINE CT) and combinations of these techniques.

In a specific embodiment of myocardial perfusion imaging, the invention relates to a method of diagnosing the presence and assessing the severity of coronary artery disease in a human patient having a history of asthma or bronchospasm. The method includes:

(a) administering by an intravenous route to the human patient from about 0.1 to about 10 μg/kg of binodenoson to provide coronary artery dilation;

(b) administering a radiopharmaceutical agent to the human patient; and

(c) performing scintigraphy on the human patient to detect the coronary artery disease.

For instance, in certain embodiments, binodenoson is administered to the human patient by an intravenous bolus dose of, for example, 1.5 μg/kg, followed by a short period, e.g., about 3 minutes, to allow coronary vasodilation, to be achieved. Then, the radiopharmaceutical agent is administered to the human patient and the scintigraphy is performed.

In other embodiments, the myocardial dysfunction is detected by ventricular function imaging. The imaging can be performed by techniques such as echocardiography, contrast ventriculography and radionuclide angiography. In the case of radionuclide angiographic studies, the studies may be first pass or gated equilibrium studies of the right and/or left ventricle.

In a specific embodiment of ventricular function imaging, the invention relates to a method of diagnosing ventricular dysfunction in a human patient having a history of asthma or bronchospasm by echocardiography. The method includes:

(a) administering by an intravenous route to the human patient from about 0.1 to 10 μg/kg of binodenoson in order to provide coronary artery dilation;

(b) performing echocardiography on the human patient to detect the ventricular dysfunction.

The echocardiography can be used, for instance, to assess the presence of abnormalities of regional wall motion and myocardial perfusion.

Invasive procedures that use binodenoson as a pharmacological stressor include those procedures where an intracardiac catheter to assess the functional significance of myocardial perfusion abnormalities. For instance, intravascular ultrasound catheters can be inserted within a coronary vessel to detect blood flow changes within the coronary vessels.

In certain embodiments, the invention relates to methods of diagnosing abnormalities in myocardial perfusion in a human patient having a history of asthma or bronchospasm. The method includes:

(a) administering by an intravenous route to the human patient from about 0.1 to 10 μg/kg of binodenoson to provide coronary artery dilation;

(b) detecting perfusion abnormalities.

In a specific embodiment of this method, the detection of perfusion abnormalities is conducted by measuring coronary blood flow velocity on the human patient to assess the vasodilatory capacity of diseased coronary vessels as compared with disease free coronary vessels.

In another specific embodiment, the detection of perfusion abnormalities is conducted by measuring coronary blood flow velocity on the human patient in order to assess the vasodilatory capacity of diseased coronary vessels as compared with disease free coronary vessels. In a particular embodiment of this method, the coronary blood flow velocity can be assessed by using a intravascular flow catheter (e.g., a Doppler flow catheter) in order to assess the vasodilatory capacity (reserve capacity) of the coronary vessels.

In specific embodiments, the detection methods of the invention may further include the step of administering an adenosine antagonist to reverse any unpleasant side effects experienced by the patient, or to more rapidly reverse the vasodilatory and the hemodynamic responses to binodenoson.

Modes of Administration: In the methods of the invention, binodenoson is administered to human patients with a history of asthma or bronchospasm by intravenous injection at a dose of about 0.1 to about 10 μg/kg. In specific embodiments, the intravenous dose is from 0.1 to 10 μg/kg. The administration can be conducted by a bolus injection or by infusion of binodenoson over time. As used herein, including the claims, “bolus dosing/administration/injection” means an injection of binodenoson over the course of no more than about 30 seconds, whereas “infusion dosing/administration/injection” means administration of binodenoson over the course of more than about 30 seconds.

In preferred embodiments of the methods of the invention, binodenoson is administered by intravenous bolus injection of vasodilatory doses of about 0.1 to about 10 μg/kg of binodenoson. Among other things, a bolus injection can obviate the need for use of an infusion pump. Preferably, the bolus dose of binodenoson administered is less than about 2.5 μg/kg, e.g., from about 0.5 to about 2.5 μg/kg, such as from about 1 to about 2 μg/kg. In certain specific embodiments, the bolus dose is less than 2.5 μg/kg, preferably from about 0.5 to about 1.5 μg/kg, more preferably about 1.5 μg/kg.

In other embodiments of the methods, where binodenoson is administered by infusion dosing. Typically, the infusion dosage is from about 0.1 to about 10 μg/kg/min and is preferably from about 0.3 to about 2.0 μg/kg/min, such as from about 0.3 to about 0.5 μg/kg/min. Generally, the infusion of binodenoson into the human patient is completed within a time period that is less than 10 minutes and, in a specific embodiment, is completed within a time period of less than 5 minutes.

Various alternative modes of administration of the adenosine A_{2A} agonists are also contemplated. These modes include administration in a parenteral dosage form, a sublingual or buccal dosage form, or administration by a transdermal device at a rate sufficient to cause vasodilation.

Kits of Administration: The invention encompasses kits that can simplify the steps needed by the clinician to effect coronary vasodilation in the human patient and/or to conduct the detection method.

A typical kit of the invention comprises a unit dosage of the adenosine A_{2A} agonist, e.g., binodenoson. In one embodiment, the unit dosage is in a container, which can be sterile, containing an effective amount of the adenosine A_{2A} agonist. In this instance, the kit can further have a second container which contain an imaging agent, an adenosine antagonist (e.g., aminophylline) or a β-2 agonist (e.g., albuterol). The imaging agent can be included in detection methods that utilize imaging procedures discussed above. Adenosine antagonists can be included in the kit as a precautionary measure to rapidly reverse the coronary hyperemic effects to the adenosine A_{2A} agonists. β-2 agonists can be included in the kit as a precautionary measure to reverse any bronchoconstriction that may be observed in asthmatic patients during or subsequent to the diagnostic procedure.

In some embodiments, the kit may further comprise an apparatus for administering the adenosine A_{2A} agonist, e.g., binodenoson, by bolus or infusion dosing. Such apparatus may include, e.g, a syringe for bolus injection of the A_{2A} agonist or an infusion pump suitable for infusion dosing of the A_{2A} agonist.

The invention is illustrated, but not limited by the following examples.

### Example 1

### Measurement of Pulmonary Responses to Binodenoson in Human Patients with Mild, Intermittent Asthma

Methodology: The study consisted of 2 parts, a Single-Blind Part and a Double-Blind Part. The dose escalating, Single-Blind Part enrolled subjects with mild, intermittent asthma and consisted of 3 sequentially enrolled dosing cohorts with 8 subjects per cohort, such that Dosing Cohorts 1, 2 and 3 received binodenoson target doses of 0.5 μg/kg, 1.0 μg/kg and 1.5 μg/kg, respectively. All 8 subjects in a dosing cohort must have completed dosing at the assigned dose and a medical review of each cohort must have been acceptable before enrollment in the next cohort began. The Double-Blind Part was initiated only if the medical review of all safety data from the Single-Blind Part was acceptable. In the Double Blind Part, subjects with mild, intermittent asthma were randomly assigned in a 2:1 ratio to receive either binodenoson 1.5 μg/kg (n=40 planned) or placebo control (n=20 planned).

Both study parts were comprised of Screening, Treatment and Follow-Up Visits. The Screening Visit occurred 7 to 14 days before the Treatment Visit and consisted of a physical examination, medical history and application of inclusion and exclusion criteria. Subjects were instructed to measure peak expiratory flow (PEF) and asthma symptoms during a period of at least 7 days prior to the Treatment Visit. Subjects were to continue to meet all Screening eligibility criteria at the Treatment Visit and forced expiratory volume in 1 second (FEV₁) was to remain within 80% of predicted for subjects to be eligible for dosing. All subjects enrolled in the Single-Blind Part and subjects randomized to receive binodenoson in the Double-Blind Part received 3 intravenous (IV) injections during the Treatment Visit: (1) placebo; (2) a low, challenge binodenoson dose to detect potential hypersensitivity reactions; and (3) an assigned binodenoson test dose. Subjects randomized to receive placebo in the Double Blind Part received 3 placebo injections. The Follow-Up Visit occurred 2 to 4 days following the Treatment Visit.

Subjects Selected For The Study: Planned enrollment was for up to 84 subjects, 24 subjects in the Single-Blind Part (3 dose escalation cohorts with 8 subjects per cohort) and 60 subjects in the Double-Blind Part (40 subjects in the binodenoson treatment group and 20 subjects in the placebo treatment group).

Analyzed: 24 subjects in the Single-Blind Part (3 dose escalation cohorts with 8 subjects per cohort) and 63 subjects in the Double-Blind Part (41 subjects in the binodenoson group and 22 subjects in the placebo group).

Eligible subjects were males or non-pregnant, nonlactating females ≧18 years of age weighing <350 pounds with a medical history of mild, intermittent asthma (as defined in the “Guidelines for the Diagnosis and Management of Asthma” prepared by the National Institutes of Health [NIH]) within 6 months of Screening. Alternatively, asthma could have been confirmed at Screening via reversibility of bronchoconstriction (defined as ≧12% increase in FEV₁) following 2 puffs of inhaled albuterol from a primed metered dose inhaler (MDI) (90 mg/puff) or 2.5 mg of albuterol solution delivered by nebulizer, or via a positive methacholine challenge test (MCT) (provocative concentration of methacholine that causes a 20% fall in FEV₁ [PC₂₀]<8 mg/mL). Subjects must have been able to control their asthma using β₂-agonists alone; been in general good, stable health as confirmed by physical examination and clinical laboratory tests; had the ability to perform reproducible pulmonary function tests (PFTs) as described by the American Thoracic Society (ATS) criteria; been a nonsmoker for at least 1 year prior to study initiation with a smoking history of ≦10 pack-years; and had a low or very low likelihood of coronary artery disease (CAD), as determined by American College of Cardiology (ACC)/American Heart Association (AHA) guidelines.

Subjects were not eligible for the study if they had resting sitting systolic blood pressure (SBP)<100 or >140 mmHg, diastolic blood pressure (DBP)<60 or >90 mmHg, pulse rate >95 beats per minute (bpm), or a lower limit FEV1 at rest of <80% of the predicted value at Screening. In addition, subjects were not eligible at the Treatment Visit if they had ≧20% variability in PEF values on ≧3 of 7 days prior to the Treatment Visit, if they had a cold, flu, or upper respiratory infection within 4 weeks prior to the Treatment Visit, or if they had a history of allergic reaction to adenosine or dipyridamole.

Dose and Mode of Administration: A stock solution of binodenoson (25 μg/mL) was administered as a bolus injection over 30 seconds (0.1 μg/kg=0.004 mL/kg of stock solution, 0.5 μg/kg=0.02 mL/kg of stock solution, 1.0 μg/kg=0.04 mL/kg of stock solution and 1.5 μg/kg=0.06 mL/kg of stock solution).

Duration of Treatment: Each subject received three 30-second bolus IV injections separated by ≧90 minutes in both the Single Blind and Double-Blind Parts of the study.

Reference Therapy, Dose and Mode of Administration, Batch Number: Placebo to match binodenoson solution was administered as a bolus injection over 30 seconds.

Criteria for Evaluation: The primary safety endpoint was clinically significant bronchoconstriction, defined as a ≧20% decrease in FEV₁ from the predrug baseline following binodenoson administration. Other safety assessments included need for rescue medication, regular measurements of pulmonary function (FEV₁ [% predicted], forced vital capacity [FVC] and forced expiratory flow during the middle half of the FVC [FEF_{25%-75%}]), vital signs, pulse oximetry, physical examination findings, electrocardiogram (ECG) results, clinical laboratory results and adverse events.

Statistical Methods: All data collected in the study were summarized by treatment (placebo; 0.1 μg/kg binodenoson challenge; or 0.5, 1.0, or 1.5 μg/kg binodenoson) and study Part (Single-Blind or Double-Blind with univariate summaries). Continuous variables were summarized with descriptive statistics (n, mean, median, standard deviation [SD] and minimum and maximum). The percent coefficient of variation was also computed, where appropriate. Categorical variables were summarized by presenting the number and percent of subjects in each category.

The safety analyses focused primarily on pulmonary reaction to binodenoson, as measured by FEV₁ change from baseline values over time. Data from secondary pulmonary measurements (e.g., change from baseline FVC, need for rescue medication) and non-pulmonary safety measurements (e.g., blood pressure, pulse rate, pulse oximetry, ECG changes, clinical laboratory results) were summarized. Treatment emergent AEs were summarized by treatment group according to the following categories: overall subject, system organ class and individual AE.

Results of the Single Blind Part of the Study: Table 4 shows the of the observed FEV₁ (as L and % predicted) following the first and second injections in the single blind part of the study. Table 5 shows the observed FEF_{25-75%} and FVC following the first and second injections in the single blind part of the study.

Table 6 shows the observed PFT parameters [FEV₁ (L and % predicted) FEF_{25-75%} and FVC] following the third injection in the single blind part of the study.

No clinically significant changes from baseline in mean FEV₁, mean FEV₁ (% predicted), mean FEF_{25%-75%}, or mean FVC were observed after the placebo or binodenoson 0.1 μg/kg injections in the Single-Blind Part (Tables 4 and 5). Moreover, no clinically significant changes from baseline in mean FEV₁, mean FEV₁ (% predicted), mean FEF_{25% 75%}, or mean FVC were observed after the binodenoson injections (third injection) during the Single-Blind Part (Table 6).

TABLE 4

| | First Injection (Placebo) | | | | Second Injection (Binodenoson 0.1 μg/kg) | | | |
|---|---|---|---|---|---|---|---|---|
| Parameter | n | Baseline | 15 minutes | 90 minutes | n | Baseline¹ | 15 minutes | 90 minutes |
| FEV₁ (L) | | | | | | | | |
| Cohort 1 | 8 | | | | 8 | | | |
| Mean (SD) | | 3.949 (0.810) | 3.919 (0.808) | 3.908 (0.834) | | 3.908 (0.834) | 3.908 (0.824) | 4.101 (0.867) |
| Mean % Change from Baseline | | | −0.759 (3.087) | −1.184 (3.382) | | | 0.136 (3.922) | 5.043 (4.097) |
| (SD) | | | | | | | | |
| Cohort 2 | 8 | | | | 8 | | | |
| Mean (SD) | | 3.779 (0.784) | 3.755 (0.899) | 3.886 (0.842) | | 3.886 (0.842) | 3.944 (0.895) | 3.895 (0.854) |
| Mean % Change from Baseline | | | −1.147 (5.579) | 2.789 (5.536) | | | 1.292 (2.662) | 0.165 (4.367) |
| (SD) | | | | | | | | |
| Cohort 3 | 8 | | | | 8 | | | |
| Mean (SD) | | 3.279 (0.671) | 3.261 (0.683) | 3.318 (0.668) | | 3.318 (0.668) | 3.325 (0.690) | 3.343 (0.657) |
| Mean % Change from Baseline | | | −0.629 (2.958) | 1.236 (2.083) | | | 0.145 (2.786) | 0.901 (2.441) |
| (SD) | | | | | | | | |
| FEV₁ (% predicted) | | | | | | | | |
| Cohort 1 | 8 | | | | 8 | | | |
| Mean (SD) | | 91.5 (11.9) | 90.9 (12.3) | 90.4 (12.1) | | 90.4 (12.1) | 90.5 (12.1) | 94.9 (12.3) |
| Mean % Change from Baseline | | | −0.6 (2.7) | −1.1 (3.3) | | | 0.1 (3.6) | 4.5 (3.5) |
| (SD) | | | | | | | | |
| Cohort 2 | 8 | | | | 8 | | | |
| Mean (SD) | | 90.6 (7.7) | 90.0 (12.0) | 93.4 (11.7) | | 93.4 (11.7) | 94.8 (13.2) | 93.6 (13.0) |
| Mean % Change from Baseline | | | −0.6 (5.0) | 2.8 (5.2) | | | 1.4 (2.5) | 0.3 (4.2) |
| (SD) | | | | | | | | |
| Cohort 3 | 8 | | | | 8 | | | |
| Mean (SD) | | 89.3 (6.4) | 88.9 (8.4) | 90.4 (7.1) | | 90.4 (7.1) | 90.5 (8.5) | 91.3 (7.3) |
| Mean % Change from Baseline | | | −0.4 (2.9) | 1.1 (1.6) | | | 0.1 (2.7) | 0.9 (2.0) |
| (SD) | | | | | | | | |

¹The 90-minute measurement for placebo served as the baseline measurement.

TABLE 5

| | First Injection (Placebo) | | | | Second Injection (Binodenoson 0.1 μg/kg) | | | |
|---|---|---|---|---|---|---|---|---|
| Parameter | n | Baseline | 15 minutes | 90 minutes | n | Baseline¹ | 15 minutes | 90 minutes |
| FEF_{25%-75%} | | | | | | | | |
| Cohort 1 | 8 | | | | 8 | | | |
| Mean (SD) | | 2.950 (0.919) | 2.970 (0.876) | 2.994 (0.939) | | 2.994 (0.939) | 2.994 (0.911) | 3.241 (0.946) |
| Mean % Change from | | | 1.131 (6.256) | 1.427 (5.574) | | | 0.526 (7.433) | 9.136 (9.019) |
| Baseline (SD) | | | | | | | | |
| Cohort 2 | 8 | | | | 8 | | | |
| Mean (SD) | | 3.339 (1.435) | 3.464 (1.705) | 3.673 (1.659) | | 3.673 (1.659) | 3.665 (1.555) | 3.471 (1.285) |
| Mean % Change from | | | 1.508 (12.796) | 8.951 (14.976) | | | 0.724 (4.318) | −2.574 (12.173) |
| Baseline (SD) | | | | | | | | |
| Cohort 3 | 8 | | | | 8 | | | |
| Mean (SD) | | 2.603 (0.678) | 2.604 (0.671) | 2.744 (0.695) | | 2.744 (0.695) | 2.758 (0.695) | 2.836 (0.636) |
| Mean % Change from | | | 0.709 (9.551) | 5.755 (3.191) | | | 0.635 (6.670) | 4.324 (8.478) |
| Baseline (SD) | | | | | | | | |
| FVC | | | | | | | | |
| Cohort 1 | 8 | | | | 8 | | | |
| Mean (SD) | | 5.606 (1.114) | 5.554 (1.131) | 5.521 (1.149) | | 5.521 (1.149) | 5.516 (1.130) | 5.654 (1.188) |
| Mean % Change from | | | −1.048 (1.873) | −1.685 (3.067) | | | −0.003 (2.480) | 2.379 (3.221) |
| Baseline (SD) | | | | | | | | |
| Cohort 2 | 8 | | | | 8 | | | |
| Mean (SD) | | 5.070 (1.162) | 4.928 (1.175) | 5.004 (1.135) | | 5.004 (1.135) | 5.120 (1.195) | 5.100 (1.219) |
| Mean % Change from | | | −2.983 (1.167) | −1.180 (2.041) | | | 2.129 (3.295) | 1.624 (3.599) |
| Baseline (SD) | | | | | | | | |
| Cohort 3 | 8 | | | | 8 | | | |
| Mean (SD) | | 4.383 (0.865) | 4.356 (0.926) | 4.340 (0.847) | | 4.340 (0.847) | 4.364 (0.876) | 4.334 (0.850) |
| Mean % Change from | | | −0.884 (4.391) | −0.930 (3.311) | | | 0.480 (2.084) | −0.163 (0.894) |
| Baseline (SD) | | | | | | | | |

¹The 90-minute measurement for placebo served as the baseline measurement.

TABLE 6

| Parameter | n | Baseline¹ | 5 min | 15 min | 45 min | 90 min |
|---|---|---|---|---|---|---|
| FEV₁ (L) | | | | | | |
| Binodenoson 0.5 μg/kg | 8 | | | | | |
| Mean (SD) | | 4.101 (0.867) | 3.995 (0.820) | 4.049 (0.800) | 4.034 (0.821) | 4.000 (0.840) |
| Mean % Change from | | | −2.327 (3.977) | −0.967 (2.171) | −1.455 (2.494) | −2.365 (3.864) |
| Baseline (SD) | | | | | | |
| Binodenoson 1.0 μg/kg | 8 | | | | | |
| Mean (SD) | | 3.895 (0.854) | 3.889 (0.883) | 3.899 (0.884) | 3.926 (1.012) | 4.005 (0.983) |
| Mean % Change from | | | −0.071 (5.830) | 0.113 (3.892) | 0.344 (6.156) | 2.406 (4.249) |
| Baseline (SD) | | | | | | |
| Binodenoson 1.5 μg/kg | 8 | | | | | |
| Mean (SD) | | 3.343 (0.657) | 3.344 (0.610) | 3.330 (0.666) | 3.331 (0.682) | 3.365 (0.680) |
| Mean % Change from | | | 0.336 (2.702) | −0.380 (2.306) | −0.439 (2.820) | 0.617 (1.762) |
| Baseline (SD) | | | | | | |
| FEV₁ (% predicted) | | | | | | |
| Binodenoson 0.5 μg/kg | 8 | | | | | |
| Mean (SD) | | 94.9 (12.3) | 92.6 (12.5) | 94.0 (11.9) | 93.4 (11.7) | 92.9 (12.5) |
| Mean % Change from | | | −2.3 (3.4) | −0.9 (2.1) | −1.5 (2.1) | −2.0 (3.4) |
| Baseline (SD) | | | | | | |
| Binodenoson 1.0 μg/kg | 8 | | | | | |
| Mean (SD) | | 93.6 (13.0) | 93.5 (11.5) | 93.6 (13.0) | 94.3 (16.4) | 96.3 (15.9) |
| Mean % Change from | | | −0.1 (5.7) | 0.0 (3.8) | 0.6 (6.3) | 2.6 (4.5) |
| Baseline (SD) | | | | | | |
| Binodenoson 1.5 μg/kg | 8 | | | | | |
| Mean (SD) | | 91.3 (7.3) | 91.6 (8.1) | 90.8 (7.8) | 90.9 (7.9) | 91.9 (7.4) |
| Mean % Change from | | | 0.4 (2.6) | −0.5 (2.0) | −0.4 (2.3) | 0.6 (1.3) |
| Baseline (SD) | | | | | | |
| FEF_{25%-75%} | | | | | | |
| Binodenoson 0.5 μg/kg | 8 | | | | | |
| Mean (SD) | | 3.241 (0.946) | 3.111 (0.915) | 3.169 (0.885) | 3.158 (0.899) | 3.106 (0.892) |
| Mean % Change from | | | −3.549 (6.717) | −1.773 (3.375) | −2.338 (5.471) | −3.732 (9.304) |
| Baseline (SD) | | | | | | |
| Binodenoson 1.0 μg/kg | 8 | | | | | |
| Mean (SD) | | 3.471 (1.285) | 3.571 (1.546) | 3.534 (1.364) | 3.759 (1.554) | 3.830 (1.595) |
| Mean % Change from | | | 2.938 (17.701) | 1.896 (7.770) | 7.715 (11.942) | 9.498 (11.643) |
| Baseline (SD) | | | | | | |
| Binodenoson 1.5 μg/kg | 8 | | | | | |
| Mean (SD) | | 2.836 (0.636) | 2.880 (0.584) | 2.890 (0.645) | 2.803 (0.705) | 2.851 (0.654) |
| Mean % Change from | | | 2.411 (12.016) | 2.110 (5.540) | −1.475 (6.450) | 0.469 (2.514) |
| Baseline (SD) | | | | | | |
| FVC | | | | | | |
| Binodenoson 0.5 μg/kg | 8 | | | | | |
| Mean (SD) | | 5.654 (1.188) | 5.581 (1.151) | 5.640 (1.130) | 5.591 (1.122) | 5.600 (1.196) |
| Mean % Change from | | | −1.162 (2.708) | −0.033 (1.645) | −0.838 (3.371) | −0.963 (3.579) |
| Baseline (SD) | | | | | | |
| Binodenoson 1.0 μg/kg | 8 | | | | | |
| Mean (SD) | | 5.100 (1.219) | 5.106 (1.192) | 5.078 (1.156) | 5.020 (1.300) | 5.105 (1.262) |
| Mean % Change from | | | 0.316 (3.947) | −0.138 (3.728) | −1.792 (6.201) | −0.051 (2.348) |
| Baseline (SD) | | | | | | |
| Binodenoson 1.5 μg/kg | 8 | | | | | |
| Mean (SD) | | 4.334 (0.850) | 4.334 (0.845) | 4.281 (0.882) | 4.326 (0.872) | 4.358 (0.878) |
| Mean % Change from | | | 0.031 (2.871) | −1.335 (2.366) | −0.179 (2.938) | 0.537 (2.460) |
| Baseline (SD) | | | | | | |

¹The 90-minute measurement for binodenoson 0.1 μg/kg served as the baseline measurement.

TABLE 7

| | First Injection (Placebo) | | | | Second Injection (Binodenoson 0.1 μg/kg) | | | |
|---|---|---|---|---|---|---|---|---|
| Parameter | n | Baseline | 15 minutes | 90 minutes | n | Baseline¹ | 15 minutes | 90 minutes |
| FEV₁ (L) | | | | | | | | |
| Placebo Group | 22 | | | | 21 | | | |
| Mean (SD) | | 3.284 (0.613) | 3.203 (0.627) | 3.286 (0.652) | | 3.286 (0.652) | 3.266 (0.661) | 3.285 (0.673) |
| Mean % Change from | | | −2.569 (3.020) | −0.660 (3.949) | | | −0.655 (3.236) | −0.130 (3.880) |
| Baseline (SD) | | | | | | | | |
| Binodenoson Group | 41 | | | | 36 | | | |
| Mean (SD) | | 3.176 (0.649) | 3.118 (0.610) | 3.148 (0.611) | | 3.156 (0.580) | 3.170 (0.590) | 3.152 (0.574) |
| Mean % Change from | | | −1.647 (3.981) | −0.578 (4.396) | | | 0.421 (2.525) | −0.042 (3.348) |
| Baseline (SD) | | | | | | | | |
| FEV₁ (% predicted) | | | | | | | | |
| Placebo Group | 22 | | | | 21 | | | |
| Mean (SD) | | 92.8 (7.9) | 90.3 (7.9) | 92.7 (9.2) | | 92.7 (9.2) | 92.0 (9.0) | 92.4 (9.1) |
| Mean % Change from | | | −2.5 (3.0) | −0.7 (3.8) | | | −0.6 (2.9) | −0.2 (3.6) |
| Baseline (SD) | | | | | | | | |
| Binodenoson Group | 41 | | | | 39 | | | |
| Mean (SD) | | 88.3 (8.5) | 86.8 (8.1) | 87.6 (7.0) | | 88.0 (6.9) | 88.3 (6.9) | 87.9 (7.7) |
| Mean % Change from | | | −1.5 (3.7) | −0.7 (3.9) | | | 0.3 (2.4) | −0.1 (2.9) |
| Baseline (SD) | | | | | | | | |
| FEF_{25%-75%} | | | | | | | | |
| Placebo Group | 22 | | | | 21 | | | |
| Mean (SD) | | 2.970 (0.941) | 2.889 (0.929) | 3.056 (0.994) | | 3.056 (0.994) | 3.090 (0.946) | 3.108 (0.971) |
| Mean % Change from | | | −2.643 (6.615) | 1.208 (9.168) | | | 1.756 (5.195) | 2.258 (6.526) |
| Baseline (SD) | | | | | | | | |
| Binodenoson Group | 41 | | | | 36 | | | |
| Mean (SD) | | 2.891 (0.998) | 2.842 (0.992) | 2.897 (1.003) | | 2.919 (0.978) | 2.946 (0.973) | 2.965 (1.071) |
| Mean % Change from | | | −1.561 (7.176) | 0.384 (10.727) | | | 1.531 (5.261) | 1.181 (6.739) |
| Baseline (SD) | | | | | | | | |
| FVC | | | | | | | | |
| Placebo Group | 22 | | | | 21 | | | |
| Mean (SD) | | 4.236 (0.853) | 4.139 (0.840) | 4.170 (0.852) | | 4.170 (0.852) | 4.118 (0.907) | 4.123 (0.917) |
| Mean % Change from | | | −2.295 (2.307) | −1.307 (3.235) | | | −1.544 (3.627) | −1.460 (4.059) |
| Baseline (SD) | | | | | | | | |
| Binodenoson Group | 41 | | | | 39 | | | |
| Mean (SD) | | 4.095 (0.911) | 4.005 (0.845) | 4.039 (0.832) | | 4.050 (0.820) | 4.051 (0.824) | 4.021 (0.778) |
| Mean % Change from | | | −1.912 (4.112) | −0.951 (4.629) | | | 0.055 (2.683) | −0.457 (3.163) |
| Baseline (SD) | | | | | | | | |

¹The 90-minute measurement for placebo (first injection) served as the baseline measurement.

TABLE 8

| Parameter | n | Baseline¹ | 5 min | 15 min | 45 min | 90 min |
|---|---|---|---|---|---|---|
| FEV₁ | | | | | | |
| Placebo | 21 | | | | | |
| Mean (SD) | | 3.285 (0.673) | 3.227 (0.658) | 3.276 (0.675) | 3.306 (0.688) | 3.300 (0.698) |
| Mean % Change from | | | −1.679 (3.571) | −0.304 (3.003) | 0.585 (2.856) | 0.347 (2.728) |
| Baseline (SD) | | | | | | |
| Binodenoson 1.5 μg/kg | 39 | | | | | |
| Mean (SD) | | 3.152 (0.574) | 3.153 (0.589) | 3.137 (0.574) | 3.156 (0.598) | 3.191 (0.588) |
| Mean % Change from | | | 0.028 (3.849) | −0.405 (3.079) | 0.056 (3.888) | 1.228 (3.835) |
| Baseline (SD) | | | | | | |
| FEV₁ (% predicted) | | | | | | |
| Placebo | 21 | | | | | |
| Mean (SD) | | 92.4 (9.1) | 91.0 (9.3) | 92.4 (10.3) | 92.9 (9.5) | 92.8 (9.9) |
| Mean % Change from | | | −1.4 (3.3) | 0.0 (2.6) | 0.5 (2.7) | 0.4 (2.5) |
| Baseline (SD) | | | | | | |
| Binodenoson 1.5 μg/kg | 39 | | | | | |
| Mean (SD) | | 87.9 (7.7) | 88.0 (8.7) | 87.6 (8.5) | 88.0 (8.3) | 89.0 (7.7) |
| Mean % Change from | | | 0.1 (3.3) | −0.3 (2.6) | 0.1 (3.3) | 1.1 (3.2) |
| Baseline (SD) | | | | | | |
| FEF_{25%-75%} | | | | | | |
| Placebo | 21 | | | | | |
| Mean (SD) | | 3.108 (0.971) | 3.061 (0.991) | 3.069 (0.976) | 3.157 (1.004) | 3.095 (0.998) |
| Mean % Change from | | | −1.757 (5.426) | −1.106 (6.720) | 1.503 (6.574) | −0.421 (8.616) |
| Baseline (SD) | | | | | | |
| Binodenoson 1.5 μg/kg | 39 | | | | | |
| Mean (SD) | | 2.965 (1.071) | 2.913 (0.981) | 2.930 (1.012) | 2.940 (0.959) | 2.923 (0.963) |
| Mean % Change from | | | −0.877 (6.999) | −0.477 (6.555) | 0.388 (7.548) | −0.335 (8.198) |
| Baseline (SD) | | | | | | |
| FVC | | | | | | |
| Placebo | 21 | | | | | |
| Mean (SD) | | 4.123 (0.917) | 4.073 (0.890) | 4.142 (0.930) | 4.146 (0.924) | 4.161 (0.912) |
| Mean % Change from | | | −1.043 (3.278) | 0.440 (2.374) | 0.600 (2.689) | 1.032 (2.569) |
| Baseline (SD) | | | | | | |
| Binodenoson 1.5 μg/kg | 39 | | | | | |
| Mean (SD) | | 4.021 (0.778) | 4.022 (0.746) | 4.004 (0.755) | 4.015 (0.780) | 4.093 (0.800) |
| Mean % Change from | | | 0.186 (4.299) | −0.309 (3.272) | −0.133 (4.369) | 1.755 (3.720) |
| Baseline (SD) | | | | | | |

¹The 90-minute measurement for the second injection served as the baseline measurement.

Results of the Double Blind Part of the Study: In the Double-Blind Part of the study, no clinically significant changes from baseline in mean FEV₁, mean FEV₁ (% predicted), mean FEF_{25%-75%}, or mean FVC were observed after the first or second injections in either the binodenoson 1.5 μg/kg or placebo groups (Table 7).

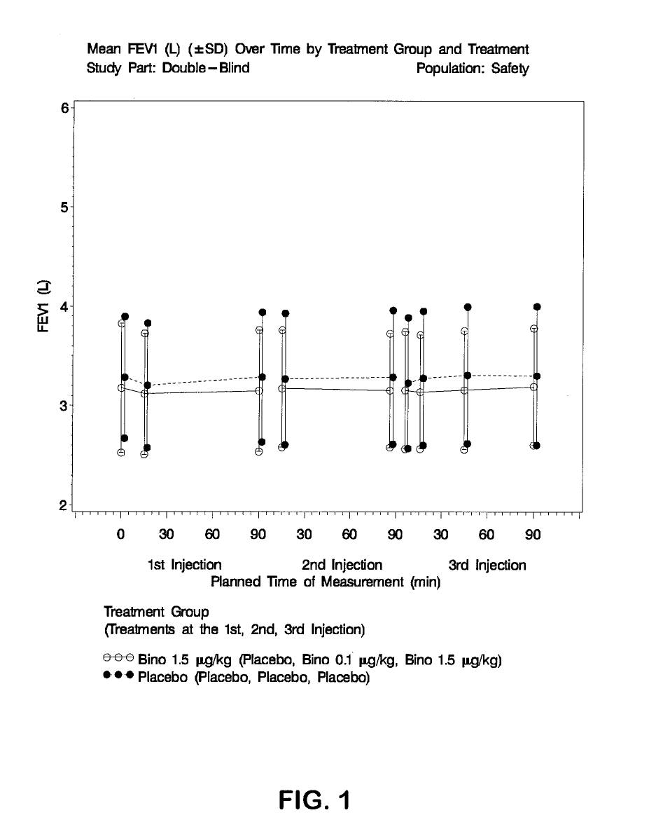

No clinically significant changes from baseline in mean FEV₁, mean FEV₁ (% predicted), mean FEF_{25% 75%}, or mean FVC were observed after the third injection (placebo or binodenoson 1.5 μg/kg) in the Double-Blind Part (Table 8). In addition, no statistically significant treatment differences were observed. FIG. 1 shows a graph of the mean FEV₁ (±SD) over time for the placebo- and binodenoson-treated patients in the Double-Blind Part of the study.

Summary of Results From Both Parts of the Study: No bronchoconstriction events were observed in either the Single- or Double-Blind Parts of the study. No subject required rescue medication during either the Single- or Double-Blind Parts of the study.

No clinically significant changes from baseline in mean FEV₁, mean FEV₁ (% predicted), mean FEF_{25%-75%}, or mean FVC were observed after any injection in either the Single- or Double-Blind Parts of the study.

No subjects experienced treatment-emergent AEs after injections of placebo or binodenoson 0.1 μg/kg in the Single-Blind Part. Half of the subjects experienced treatment-emergent AEs after the third injection in the Single-Blind Part (25% binodenoson 0.5 μg/kg, 50% binodenoson 1.0 μg/kg and 75% binodenoson 1.5 μg/kg). In the Double-Blind Part, 19% of placebo subjects and 69% of binodenoson 1.5 μg/kg subjects experienced AEs, the majority of which occurred after the third injection. The most common treatment emergent AEs in the binodenoson 1.5 μg/kg group were tachycardia (31%), dizziness (18%), flushing (15%), sinus tachycardia and nausea (8% each) and headache and abdominal discomfort (5% each). No specific AE occurred in more than 1 subject in the placebo group.

There were no deaths, serious AEs, or premature discontinuations due to AEs during the course of the study.

No clinically meaningful results were observed with respect to laboratory evaluations, ECG, or pulse oximetry. Transient increases in SBP and pulse rate and decreases in DBP were observed in both treatment groups in the Double-Blind Part, with the magnitude of changes greater in the binodenoson compared to the placebo group.

Conclusions: The results of this study in subjects with mild, intermittent asthma demonstrate that binodenoson in doses up to 1.5 μg/kg did not induce bronchoconstriction; and binodenoson in doses up to 1.5 μg/kg was safe and well tolerated; no clinically significant effects on pulmonary function parameters, laboratory evaluations, vital signs, ECG, or pulse oximetry were observed.

### Example 2

### Dosing Regimens of Binodenoson that Produce Coronary Microcirculatory Vasodilation Comparable to Adenosine in Human Patients without Histories of Asthma or COPD

This example describes studies designed to determine useful doses and dosing regimens for binodenoson use as a pharmacologic stressor. Specifically, the study was designed to establish the binodenoson dosing regimen that produced a level of coronary vasodilation comparable to that produced by adenosine during a pharmacologic stress procedure, with the fewest and least severe side effects. Coronary blood flow velocity reserve (CBFVR) was established by intracoronary (IC) bolus injections of adenosine just prior to administration of binodenoson to allow a direct comparison of the magnitude of responses.

Patients presenting for cardiac catheterization were screened for eligibility and provided informed consent prior to sedation. Final eligibility was determined by the investigator during diagnostic catheterization. Eligible patients included males or nonpregnant females aged ≧18 years and weighing between 40 and 125 kg who had at least 1 unobstructed coronary artery that was technically accessible and into which a Doppler guide wire (FloWire™, Volcano Corporation, Rancho Cordova, Calif.) could be introduced. Patients were excluded if they had ingested caffeine, methylxanthines, or dipyridamole within 12 hours or had a history of hypersensitivity to aminophylline or theophylline; had received any investigational drug within 30 days; had enrolled in a previous binodenoson study; had active asthma or chronic obstructive pulmonary disease; had an acute myocardial infarction within 30 days; had uncontrolled hypertension, congestive heart failure, left ventricular hypertrophy, dilated cardiomyopathy, malignant ventricular arrhythmias, clinically significant valvular disease, left ventricular ejection fraction ≦40%, a patent bypass graft or stent in the vessel of interest, left main coronary artery disease (>50% luminal narrowing by visual inspection), severe 3-vessel disease (>80% in 3 major vessels), angiographic appearance suggestive of thrombus, or had undergone a percutaneous intervention during catheterization.

Patients had a 12-lead electrocardiogram (ECG) within 7 days and blood drawn for clinical laboratory testing within 24 hours prior to study entry. Following completion of the diagnostic catheterization procedure and confirmation of all eligibility criteria, the Doppler guide wire was introduced into an accessible coronary artery and manipulated until a stable signal was obtained.

Drug administration: All usual catheterization procedural medications including IC nitroglycerin, heparin, anxiolytics and analgesics, were allowed. Within 15 minutes prior to administration of binodenoson, 2 to 3 escalating IC adenosine doses were injected rapidly into the target coronary artery to define CBFVR. Binodenoson doses were then introduced into a peripheral vein via an indwelling catheter. All 133 patients in the dose-selection study were randomly assigned to receive 1 of 5 intravenous (IV) dosing regimens: binodenoson by continuous infusion for 3 minutes at rates of 0.3, 0.5, or 1 μg/kg/min (total doses 0.9, 1.5 and 3 μg/kg) or binodenoson doses of 1.5 or 3 μg/kg by bolus IV injection over 30 seconds.

Measurements: Coronary blood flow velocity (CBFV) was measured as continuous pulsatile (systolic and diastolic) velocity (cm/sec) with the Doppler guide wire introduced via a guiding catheter. HR was derived from the ECG signal. SBP and diastolic blood pressure (DBP) were recorded directly from the catheter sheath. For each IC adenosine injection, CBFVR was calculated by dividing the peak post-injection CBFV value by the respective baseline CBFV value. Each patient's maximal calculated CBFVR was used as a benchmark to which CBFV responses to binodenoson were compared. For each binodenoson dose, baseline CBFV (post-IC adenosine), peak CBFV, time following dose start time to achieve peak CBFV and percents of CBFVR (calculated as the ratio of CBFV change from baseline following binodenoson administration vs CBFVR) at each point were calculated. Rate pressure product (RPP) and coronary vascular resistance (CVR) were derived at each time point (see formulas in Table 10). Patients in the study were monitored continuously until CBFV returned to baseline, for 10 minutes after CBFV returned to within 25% of pre-binodenoson baseline, or for a total of 45 minutes, whichever occurred first. Vital signs were measured again approximately 3 to 4 hours after dosing or prior to hospital discharge. Patients returned for a follow-up visit 2 to 4 days following catheterization that included vital signs, an abbreviated physical examination, a 12-lead ECG, blood chemistry and hematology assessments and an assessment of any late-emerging adverse events.

Adverse events were monitored throughout the study. A conservative approach was taken, per protocol, to identify decreases in SBP and DBP in the dose-selection study: decreases in SBP >20 mmHg or decreases in DBP >15 mmHg were to be reported as adverse events regardless of the baseline blood pressure. The incidence of clinically significant changes, defined as decreases in SBP to <80 mmHg or in DBP to <45 mmHg, were also recorded. Serious adverse events were defined as those that resulted in death; that were life-threatening or disabling; or that required or prolonged hospitalization. The Coding Symbols for Thesaurus of Adverse Reaction Terms (COSTART) dictionary (version 5.0, Food and Drug Administration, Rockville, Md.) was used to code adverse events by body system and preferred term.

Statistical analyses: All pharmacodynamic and safety data were analyzed using the intent-to-treat (ITT) population, which included all patients who received any amount of study drug. A paired t test was used to assess the significance of within-treatment differences (peak vs baseline) in CBFV, vital signs and calculated CVR and RPP; repeated comparisons were corrected using the Bonferroni-Holm method. The statistical significance of differences among treatment groups was evaluated using an analysis of variance (ANOVA) model that included treatment and investigator interactions.

Enrollment of 120 patients in the dose-selection study (24 patients per dose) provided 90% power to conclude that the lower boundary of the 95% confidence limit on the population success rate was 65%. Success was defined as coronary hyperemia that remained ≧85% of CBFVR for ≧2 minutes. To allow for dropouts, an enrollment of 138 patients was planned.

Results: In all, 138 patients were enrolled and 133 received a single dose of study drug and were included in ITT analyses. Five randomized patients did not receive the study medication because of pretreatment adverse events, technical difficulties, or withdrawal of consent. Demographic characteristics and baseline (pre-IC adenosine) CBFV values were similar across the 5 dose groups. IC adenosine produced transient increases in CBFV values but no consistent effects on SBP, DBP, or HR; patient responses to IC adenosine and mean doses of adenosine resulting in CBFVR were similar across dose groups.

TABLE 9
CBFV and Percent of CBFVR Achieved Following
Binodenoson Dosing (ITT population)*

| | Binodenoson Infusion (μg/kg/min × 3 min) | | | Binodenoson Bolus (μg/kg) | |
|---|---|---|---|---|---|
| | 0.3 | 0.5 | 1 | 1.5 | 3 |
| | (n = 26) | (n = 28) | (n = 26) | (n = 28) | (n = 25) |
| Baseline CBFV* | | | | | |
| (cm/sec) | | | | | |
| Mean ± SD | 21.3 ± 8.4 | 18.9 ± 11.0 | 18.5 ± 7.5 | 22.2 ± 10.8 | 18.3 ± 4.4 |
| Range | 8-38 | 7-57 | 8-35 | 6-55 | 13-31 |
| Peak CBFV*^{†} | | | | | |
| (cm/sec) | | | | | |
| Mean ± SD | 55.2 ± 21.0 | 49.6 ± 20.4 | 53.1 ± 14.8 | 54.0 ± 19.9 | 55.8 ± 14.6 |
| Range | 17-122 | 26-135 | 29-81 | 21-96 | 35-94 |
| Time of peak* | | | | | |
| CBFV (min) | | | | | |
| Mean ± SD | 4.3 ± 2.8 | 5.4 ± 5.9 | 5.8 ± 3.8 | 4.5 ± 3.7 | 6.0 ± 3.8 |
| Range | 1-12 | 1-30 | 1-13 | 1-15 | 1-14 |
| % of CBFVR at | | | | | |
| peak* | | | | | |
| Mean ± SD | 83.5 ± 19.4 | 95.0 ± 40.4 | 100.9 ± 22.1 | 90.6 ± 23.7 | 99.9 ± 22.1 |
| Range | 40.0-124.5 | 52.2-288.1 | 66.5-144.5 | 45.8-156.6 | 44.4-130.5 |
| Duration (min) of | | | | | |
| hyperemia ≧85% | | | | | |
| of CBFVR^{‡} | | | | | |
| Mean ± SD | 3.1 ± 1.97 | 5.3 ± 4.53 | 10.9 ± 8.54 | 7.4 ± 6.86 | 12.3 ± 9.59 |
| Range | 1-8 | 1-14 | 1-24 | 1-21 | 2-39 |

*p > 0.05 for overall treatment effect for each outcome variable (ANOVA);
^{†}Post-binodenoson. Highest CBFV after binodenoson during the in-catheterization observation period;
p < 0.001 for each within-treatment difference between peak and baseline (paired t test);
^{‡} p = 0.006 treatment effect (ANOVA);
ANOVA = analysis of variance;
CBFV = coronary blood flow velocity;
CBFVR = coronary blood flow velocity reserve (peak CBFV following IC adenosine/baseline CBFV);
cm = centimeters;
ITT = intent-to-treat;
min = minute;
SD = standard deviation;
sec = second.

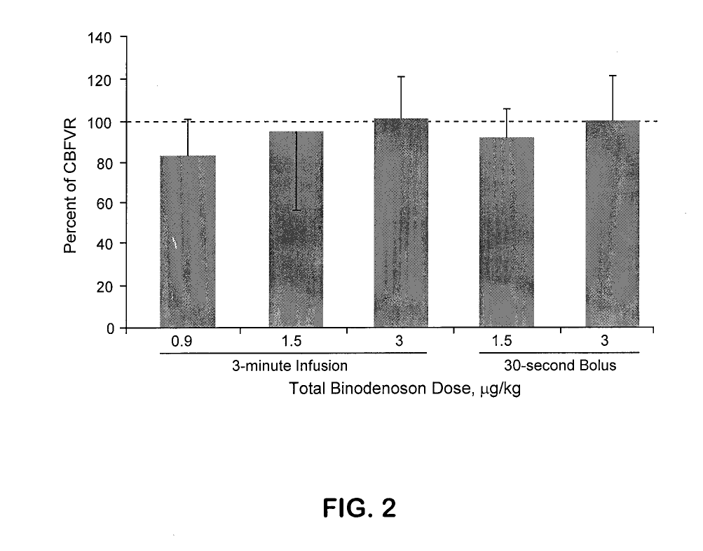

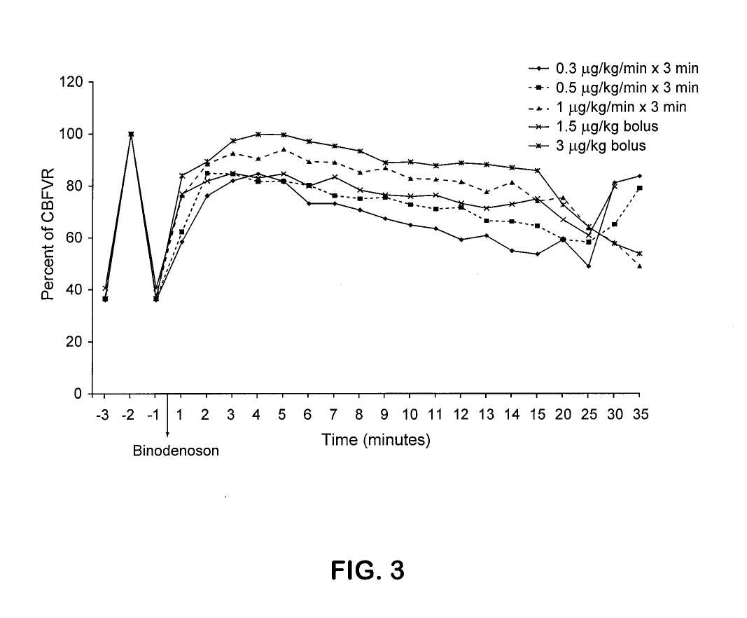

Baseline mean CBFV, HR, SBP, DBP, CVR and RPP values prior to binodenoson dosing were similar across treatment groups (Table 9). Coronary hyperemic responses to binodenoson were evident within seconds of drug administration. CBFV achieved nearly maximal levels within 3 minutes and the mean peak response occurred within the first 6 minutes in all treatment groups (p<0.001, paired t test, each group). Peak responses were similar across treatment groups (p=0.757, ANOVA; FIG. 2). Hyperemic responses within each group were significant at each time point after the start of binodenoson dosing (p<0.001, repeated measures ANOVA; FIG. 3). The 1.5 and 3 μg/kg doses, whether infused over 3 minutes or injected as boluses, produced maximal coronary hyperemia equivalent to CBFVR and the hyperemic response to the 0.3 μg/kg/min times 3-minute infusion was only slightly less effective (FIG. 2; Table 9). The duration of mean maximal hyperemia (time CBFV was ≧85% of CBFVR) was dose related (p=0.006, ANOVA; Table 9). Maximal hyperemia persisted for 7.4±6.86 minutes following the 1.5 μg/kg bolus dose. Mean CBFV responses for all 5 doses, expressed as percents of CBFVR, are presented in FIG. 3.

TABLE 10
Vital Signs and Hemodynamic Parameters Following Binodenoson Dosing (ITT population)*

| | Binodenoson Infusion (μg/kg/min × 3 min) | | | Binodenoson Bolus (μg/kg) | |
|---|---|---|---|---|---|
| | 0.3 | 0.5 | 1 | 1.5 | 3 |
| | (n = 26) | (n = 28) | (n = 26) | (n = 28) | (n = 25) |
| HR (bpm)^{†} | | | | | |
| Baseline | 74.7 ± 17.6 | 69.5 ± 14.4 | 72.04 ± 12.0 | 75.04 ± 13.8 | 74.9 ± 14.6 |
| Maximum^{‡} | 95.0 ± 17.9 | 94.5 ± 17.6 | 102.7 ± 16.6 | 97.1 ± 14.7 | 108.0 ± 11.4 |
| SBP (mmHg)^{†} | | | | | |
| Baseline | 134.5 ± 26.0 | 133.3 ± 29.9 | 128.4 ± 24.1 | 132.7 ± 23.2 | 126.0 ± 21.1 |
| Maximum | 108.6 ± 24.5 | 108.0 ± 20.4 | 105.0 ± 23.7 | 103.2 ± 20.0 | 103.2 ± 17.4 |
| DBP (mmHg)^{†} | | | | | |
| Baseline | 75.0 ± 11.8 | 73.6 ± 12.0 | 71.6 ± 10.8 | 72.2 ± 8.4 | 73.8 ± 11.0 |
| Maximum | 57.9 ± 12.9 | 58.3 ± 10.6 | 55.8 ± 10.9 | 54.6 ± 10.1 | 58.8 ± 9.9 |
| CVR^{†} | | | | | |
| Baseline | 5.3 ± 2.2 | 6.2 ± 3.3 | 5.8 ± 2.7 | 5.1 ± 2.6 | 5.2 ± 1.2 |
| Maximum | 1.7 ± 0.6 | 1.9 ± 0.7 | 1.7 ± 0.6 | 1.7 ± 0.8 | 1.6 ± 0.4 |
| RPP^{†} | | | | | |
| Baseline | 9913 ± 3051 | 9096 ± 2087 | 9111 ± 1859 | 9975 ± 2784 | 9344 ± 2009 |
| Maximum^{§} | 12035 ± 2686 | 11995 ± 2795 | 12839 ± 3355 | 12101 ± 2974 | 13152 ± 2573 |

*Mean ± standard deviation. Maximum values reflect variables at their maximum increase from baseline during the in-catheterization observation period. For each variable, p < 0.001 for each within-treatment difference between maximum and baseline (paired t test);
^{†}p > 0.05,
^{‡}p = 0.003;
^{§}p = 0.010 for overall treatment effect (ANOVA);
Formulas:
CVR (cm*mmHg/sec) = ([SBP − DBP]/3 + DBP)/CBFV
RPP (beats*mmHg/min) = SBP × HR
ANOVA = analysis of variance;
bpm = beats per minute;
CBFV = coronary blood flow velocity;
CVR = coronary vascular resistance;
DBP = diastolic blood pressure;
HR = heart rate;
min = minute;
ITT = intent-to-treat;
RPP = rate pressure product;
SBP = systolic blood pressure;
sec = second.

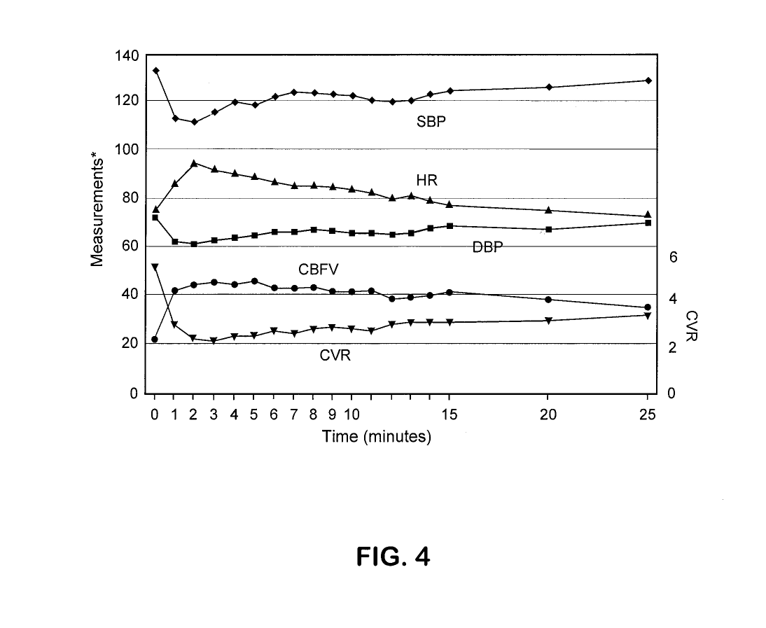

The hyperemic responses were accompanied by dose-related increases in HR (p=0.003, ANOVA) and RPP (p=0.010, ANOVA) and increases in HR and RPP were greatest in patients treated with the 3 μg/kg doses (Table 10). Modest decreases in SBP, DBP and CVR were similar across doses (p=0.42, 0.45 and 0.42, respectively, ANOVA; Table 10) and peak changes in vital signs were similar when comparable doses were administered by infusion or bolus injection. Binodenoson-induced changes in mean SBP, DBP, CVR, RPP and HR returned to near-baseline levels within approximately 15 minutes. It was not possible to determine accurately the time required for CBFV to return to baseline since catheters were removed from most patients approximately 15 minutes after dosing. All patients were stable at this time. Extrapolation of the decaying CBFV responses suggests CBFV would return fully to baseline within 30 minutes. Mean CBFV, CVR, SBP, DBP and HR responses to a 1.5 μg/kg bolus dose are illustrated in FIG. 4.

TABLE 11
Adverse Events Reported in ≧5% of Patients in
Any Treatment Group, n (%) (ITT population)*

| | Binodenoson Infusion | | | Binodenoson | |
|---|---|---|---|---|---|
| | (μg/kg/min × 3 min) | | | Bolus (μg/kg) | |
| Body System | 0.3 | 0.5 | 1 | 1.5 | 3 |
| Preferred Term | (n = 26) | (n = 28) | (n = 26) | (n = 28) | (n = 25) |
| Any adverse event | 19 (73) | 25 (89) | 23 (89) | 26 (93) | 21 (84) |
| Hypotension* | 17 (65) | 19 (68) | 13 (50) | 20 (71) | 14 (56) |
| Hypotension^{†} | 3 (12) | 2 (7) | 4 (15) | 4 (14) | 2 (8) |
| Hemorrhage | 0 | 1 (4) | 2 (8) | 3 (11) | 2 (8) |
| Vasodilation | 0 | 3 (11) | 3 (12) | 0 | 1 (4) |
| Bradycardia | 1 (4) | 0 | 2 (8) | 1 (4) | 0 |
| Abdominal pain | 0 | 0 | 0 | 1 (4) | 2 (8) |
| Back pain | 2 (8) | 5 (18) | 1 (4) | 4 (14) | 1 (4) |
| Chest pain | 0 | 1 (4) | 4 (15) | 4 (14) | 5 (20) |
| Headache | 1 (4) | 2 (7) | 2 (8) | 4 (14) | 4 (16) |
| Injection site reaction | 1 (4) | 1 (4) | 2 (8) | 0 | 0 |
| Nonspecified pain | 1 (4) | 4 (14) | 5 (19) | 3 (11) | 2 (8) |
| Nausea | 3 (12) | 3 (11) | 2 (8) | 2 (7) | 5 (20) |
| AST or ALT | 0 | 0 | 0 | 1 (4) | 2 (8) |
| increased | | | | | |
| Dizziness | 0 | 0 | 3 (12) | 2 (7) | 1 (4) |
| Dyspnea | 0 | 2 (7) | 1 (4) | 1 (4) | 1 (4) |
| Ecchymosis | 0 | 0 | 1 (4) | 0 | 2 (8) |

*SBP decreased by >20 mmHg or DBP decreased by >15 mmHg from baseline;
^{†}SBP values <80 mmHg or DBP values <45 mmHg;
ALT = alanine aminotransferase;
AST = aspartate aminotransferase;
DBP = diastolic blood pressure;
ITT = intent-to-treat;
SBP = systolic blood pressure.

All doses of binodenoson were well tolerated. Most patients experienced at least 1 adverse event (11). There was no significant difference in the overall incidence of adverse events across groups (p=0.280, Pearson's chi-square test), although those receiving the lowest dose (0.3 μg/kg/min×3 min) reported the fewest drug-related adverse events (Table 11). Most adverse events were rated as mild (84%) or moderate (15%) in intensity. Because of the protocol-defined criteria that decreases in SBP >20 mmHg or in DBP >15 mmHg be reported as adverse events, hypotension was the most commonly reported adverse events; such responses were reported by 50% to 71% of patients in each dose group and were not dose related. However, only 2 to 4 patients per group (7% to 15%) experienced decreases in SBP <80 mmHg or in DBP <45 mmHg. There were no adverse changes or trends in ECGs at any dose and there were no ECG-related adverse events during or following binodenoson administration. A list of adverse events reported by ≧5% patients is provided in Table 11.

Two serious adverse events (ventricular fibrillation [n=1], myocardial infarction [n=1]) occurred prior to treatment. Seven serious adverse events occurred in 6 patients during the study: thrombosis (n=1) and hemorrhage (n=2) were considered unrelated to study drug. Hypotension (n=2), bradycardia (n=1) and ventricular tachycardia (n=1) were considered related to study drug. The frequency was not dose related. Three patients prematurely discontinued the 1 μg/kg/minute times 3-minute infusion due to dyspnea (n=1) or hypotension (n=2).

Summary: Like adenosine, the onset of binodenoson-induced hyperemia is immediate. Maximal coronary vasodilatory responses tended to be dose related, although only the 0.9 μg/kg infusion dose produced less than maximal hyperemia. Because doubling the dose of infusions and bolus injections from 1.5 to 3 μg/kg did not produce significantly greater coronary hyperemia, the 1.5 μg/kg IV bolus dose appears to represent the upper asymptote of the hyperemic dose-effect curve. Maximal hyperemia persisted longer following the 3 μg/kg bolus (12.3±9.59 min) than the 1.5 μg/kg dose (7.4±6.86 min) but at the expense of higher HR and RPP and more adverse events. The duration of the 1.5 μg/kg hyperemic response is clearly sufficient to allow adequate extraction of ²⁰¹Tl and ^{99m}Tc-labeled radiopharmaceuticals used in single photon emission computed tomography (SPECT) imaging.

### Example 3

### Assessment of Pharmacokinetics and Safety of Binodenoson in Non-Asthmatic Human Patients

This example describes studies designed to assess single-dose pharmacokinetics, safety and tolerability of intravenous binodenoson. Binodenoson was been administered to human beings to determine the safety and pharmacokinetics of a wide range of doses.

Subjects: This study was conducted at the New Orleans Center for Clinical Research, New Orleans, La., in accordance with US Good Clinical Practice guidelines. Subjects were required to be in generally good health as determined by physical examination and laboratory testing, as well as assessment of vital signs. Exclusion criteria included testing positive on drug screening (drugs of abuse); ingesting caffeine, alcohol, or medication within 24 hours of study entry; or receiving an investigational drug with 30 days of study entry. Women of childbearing potential and men whose female partners were not using an acceptable contraceptive method were excluded. Other exclusion criteria included subjects with known postural hypotension, resting supine systolic blood pressure of 90 mmHg or lower, diastolic blood pressure of 60 mmHg or lower and heart rate of 90 beats/min or greater; history of human immunodeficiency virus infection; positive test result for hepatitis B surface antigen or hepatitis C antibody; and any clinically relevant condition that could potentially confound the analysis or present a safety risk.

Study Methods: The study was designed as a single-center, open-label, nonrandomized, intravenous dose-escalation study in 4 cohorts (n=6 each) of healthy volunteers. The protocol was approved by the institutional review board; all subjects gave written informed consent. Subjects from each cohort were to receive 3 rising doses of binodenoson administered via intravenous infusion over a period of 10 minutes, at a rate not to exceed 6 μg/kg/min. Although the successive doses of binodenoson were administered on the same study day, the washout period between doses was at least 2 hours. Subjects in cohort 1 received consecutive binodenoson doses of 0.1, 0.2 and 0.4 μg/kg; cohort 2 received 0.6, 1 and 2 μg/kg; cohort 3 received 2, 3 and 4 μg/kg; and cohort 4 received 4, 5 and 6 μg/kg.

Serial vital sign monitoring of heart rate, supine systolic blood pressure and diastolic blood pressure was done at screening and during each dosing phase within 10 minutes before infusion, at 2, 4, 6, 8 and 10 minutes during infusion and at 2, 5, 7.5, 10, 15, 20, 30, 45, 60, 90 and 120 minutes after infusion. A 12-lead electrocardiogram (ECG) was obtained at screening and the end of the study. The ECG was monitored via telemetry during the treatment phase, including during the infusions. Monitoring was initiated within 1 hour before dosing and continued until 24 hours after completion of the last dose.

A total of 40 to 42 blood samples were collected during the treatment phase for quantitation of binodenoson in plasma. Before dosing, a polyethylene catheter was inserted into a vein of the forearm contralateral to the infusion site. Blood samples (5 mL) were drawn into prechilled Vacutainer tubes (BD, Franklin Lakes, N.J.) just before infusion (−1 minute), at the midpoint (5 minutes) and end (10 minutes) of infusion and at 2, 5, 7.5, 10, 15, 20, 30, 45, 60, 90 and 120 minutes after the end of infusion. Plasma was separated from cellular material by centrifugation under refrigeration (4° C.) at 4000 rpm for 10 minutes and then stored in cryotubes at −80° C. until analyzed. The total amount of blood taken during the intensive sampling period was approximately 200 mL. Plasma concentrations of binodenoson were determined by a validated high-performance liquid chromatography-mass spectrometry (LC/MS/MS) assay at Phoenix International, Inc. (Montreal, Quebec, Canada). The LC/MS/MS assay had a lower limit of quantitation of 0.201 ng/mL.

Pharmacokinetic parameters were derived for each subject's plasma binodenoson concentration-time profile for each binodenoson infusion by use of noncompartmental methods with the statistical software program SAS (SAS Institute, Cary, N.C.). The peak concentration (Cₘₐₓ) and corresponding time to (Cₘₐₓ) (tₘₐₓ) were derived by observation. The terminal half-life (t_{1/2}) was calculated from (ln 2)/λ_{z}, where λ_{z}, the elimination rate constant, was determined by log-linear regression of the terminal phase of the binodenoson concentration-time profile. The area under the curve (AUC₀₋ₜ) was calculated by the linear trapezoidal rule from time 0 to the last quantifiable concentration (Cₗₐₛₜ). The area up to infinity (AUC_{0-∞}) was estimated by summation of AUC₀₋ₜ+Cₗₐₛₜ/λ_{z}. Systemic clearance (CL) of binodenoson was derived from the ratio of binodenoson dose and AUC_{0-∞} and volume of distribution (V_{z}) was derived from the ratio of CL and λ_{z}. Summary statistics for pharmacokinetic parameters were tabulated. Linear regression analysis was used to evaluate the relationship between AUC and dose.

The safety analysis focused on vital signs, physical examination findings, clinical laboratory values, ECGs and AEs. AEs and serious AEs were defined according to US Food and Drug Administration regulations. AEs recorded were those reported spontaneously by volunteers or in response to nonleading questions or those recognized by investigators. Safety data were tabulated by cohort and/or dose. The maximal effect of binodenoson dose on vital signs (systolic blood pressure, diastolic blood pressure, heart rate) was analyzed by comparing the predose mean value with the maximally changed value recorded during each 10-minute infusion period or during the 120-minute postinfusion period. A 2-tailed, paired Student t test was used to determine whether the change was significantly different from 0 (α=0.05).

Subjects: A total of 24 healthy adult volunteers (17 men and 7 women) participated in the study. The mean values for age and weight over all cohorts ranged from 29 to 39 years and 70.8 to 83.3 kg, respectively. For the study group as a whole, the mean age was 35±9 years and the mean body weight was 75.6±12.0 kg. Of the subjects, 15 of 24 (63%) were white, 8 were black and 1 was Hispanic. All enrolled subjects met the study inclusion and exclusion criteria and no concomitant medications were used during the study.

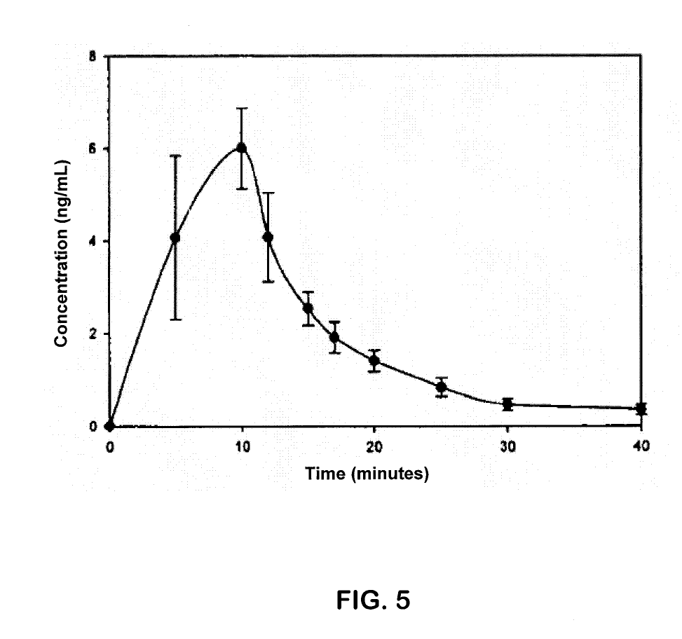

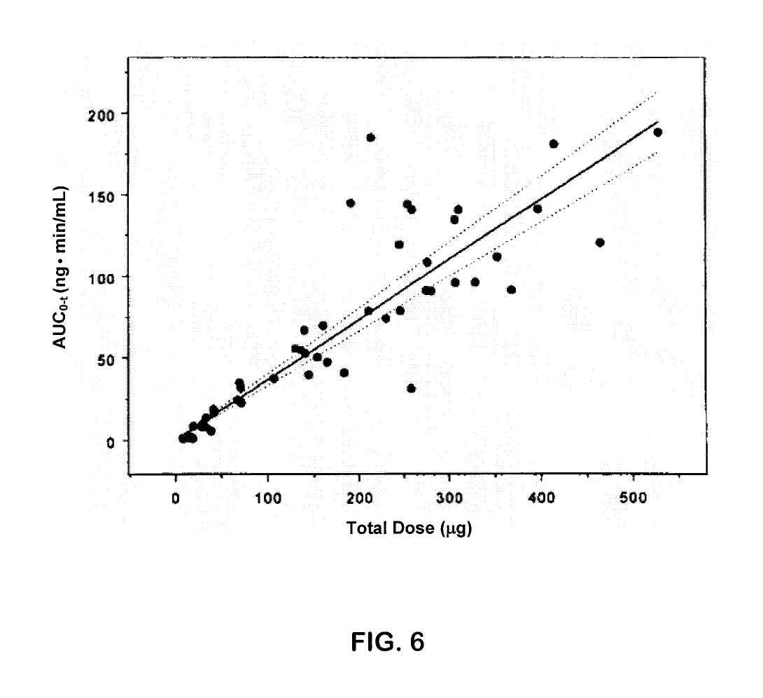

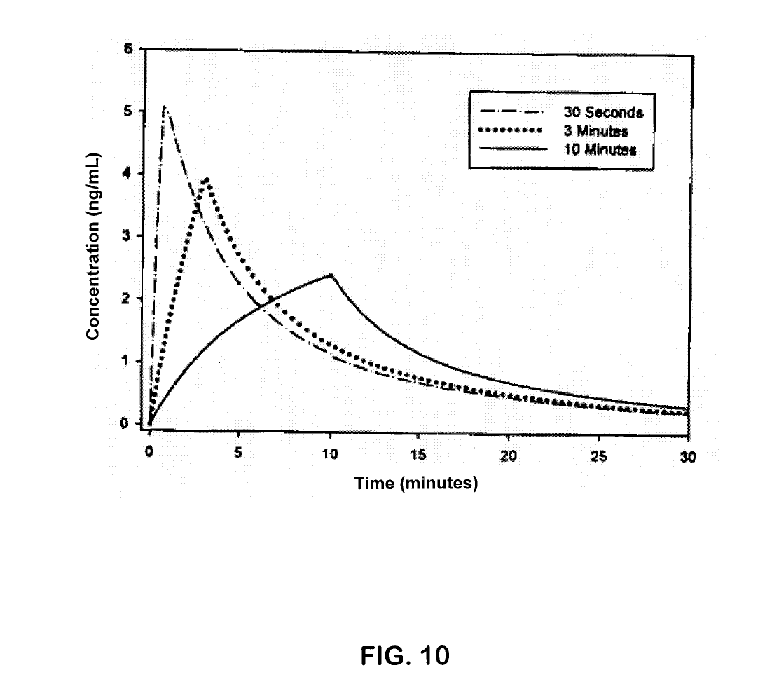

Pharmacokinetics: The pharmacokinetics of binodenoson is presented in Table 12. Peak concentrations (Cₘₐₓ) were generally achieved by the end of the dosing infusion period (FIGS. 5 and 10). Thereafter binodenoson concentrations declined in a biphasic manner. Area under the curve as calculated by the trapezoidal rule (AUC₀₋ₜ) generally represented greater than 80% of the total AUC_{0-∞}. Binodenoson AUC increased proportionally with dose (FIG. 6). Binodenoson Cₘₐₓ also increased with dose but was subject to change resulting from slight variations in the duration of infusion. The apparent volume of distribution indicates that binodenoson distributes into extracellular fluid spaces.

TABLE 12

| | Dose of Binodenoson (μg/kg) | | | | | | | |
|---|---|---|---|---|---|---|---|---|
| | 0.4 | 0.6 | 1 | 2 | 3 | 4 | 6 | 8 |
| n | 6 | 6 | 6 | 10 | 6 | 11 | 6 | 4 |
| Cₘₐₓ (ng/mL) | 0.9 (0.2) | 1.1 (0.1) | 2.1 (0.5) | 3.9 (0.9) | 6.2 (0.8) | 8.7 (3.9) | 12.5 (2.7) | 12.1 (4.3) |
| Tₘₐₓ (min) | 10 | 7.5 | 10 | 10 | 9.5 | 10 | 9 | 10.5 |
| AUCₗₐₛₜ (ng · min/mL) | 9.0 (2.6) | 12.5 (5.3) | 24.9 (7.4) | 51.3 (10) | 78.7 (7.2) | 116 (40) | 133 (15) | 156 (44) |
| AUC_{0-∞}(ng · min/mL) | 12.4 (2.6) | 21.2 (3.6) | 27.9 (8.2) | 55.7 (9.7) | 85.4 (7.6) | 122 (40) | 139 (15) | 163 (44) |
| T_{1/2} (min) | 8.8 (8.7) | 11.7 (6.3) | 7.4 (1.8) | 10.0 (2.3) | 12.8 (3.8) | 13.0 (3.1) | 14.9 (3.3) | 12.3 (2.2) |
| CL (mL/min/kg) | 33.4 (6.7) | 28.8 (4.4) | 38.3 (10) | 33.0 (12) | 35.4 (3.3) | 39.8 (27) | 33.9 (7.9) | 39.5 (14) |
| V_{z} (L/kg) | 0.39 (0.3) | 0.46 (0.2) | 0.40 (0.2) | 0.48 (0.2) | 0.65 (0.2) | 0.69 (0.3) | 0.70 (0.1) | 0.68 (0.7) |

Data are given as mean (SD).

The mean values for apparent elimination half-life of binodenoson ranged from 7.4 minutes (at 1 μg/kg) to 14.9 minutes (6 μg/kg), with a slight tendency toward higher values with increasing dose. However, because the plasma concentrations for the lowest dose level (0.4 μg/kg) were only marginally higher than the assay lower limit of quantitation, the plasma concentrations could be determined for a longer period of time at the higher dose levels. On average (harmonic mean), the terminal half-life of binodenoson across all doses was 10±4 minutes.

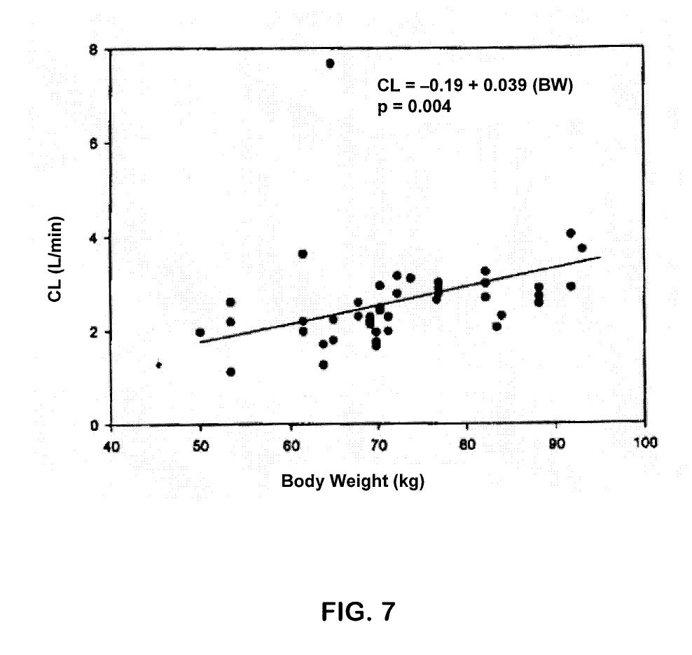

There was no detectable difference (analysis of variance) among the dose levels with respect to binodenoson systemic clearance. The mean systemic clearance of binodenoson across all dose levels was 34.4±7.5 mL/min/kg. However, systemic clearance correlated with subject body weight as shown in FIG. 7. A linear mixed-effect model (S-Plus, Insightful Corp., Seattle, Wash.), with subject used as a random-effects variable and body weight as a regressor term, established the following relationship between binodenoson clearance (CL) and body weight (BW): CL=−0.19+0.039 BW (p=0.004).

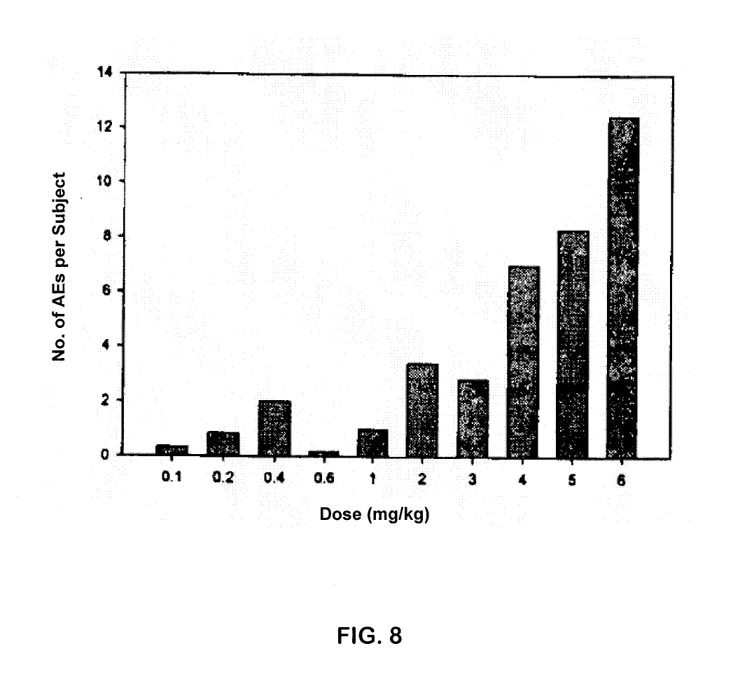

Adverse Effects: Generally, binodenoson was well tolerated. There were no serious AEs and no clinically significant ECG changes. The incidence of AEs was dose-related and 21 of 24 volunteers (83%) reported at least 1 AE (FIG. 8). Nearly all AEs (99%) were judged by the investigator to be related to administration of binodenoson and were of mild (82%) or moderate (17%) severity. Most AEs (75%) began during drug infusions and resolved spontaneously within 30 minutes of onset. None required clinical or pharmacologic intervention to reverse the action of the drug. The most frequently reported AEs were headache and vasodilation. There was a significant increase in frequency of AEs related to binodenoson at doses of 2 μg/kg or greater as compared with doses of 1 μg/kg or lower, particularly with respect to headache (60% vs 7%), nausea/vomiting (49% vs 0%), vasodilation (54% vs 14%), dizziness (24% vs 0%) and paresthesia (19% vs 3%).

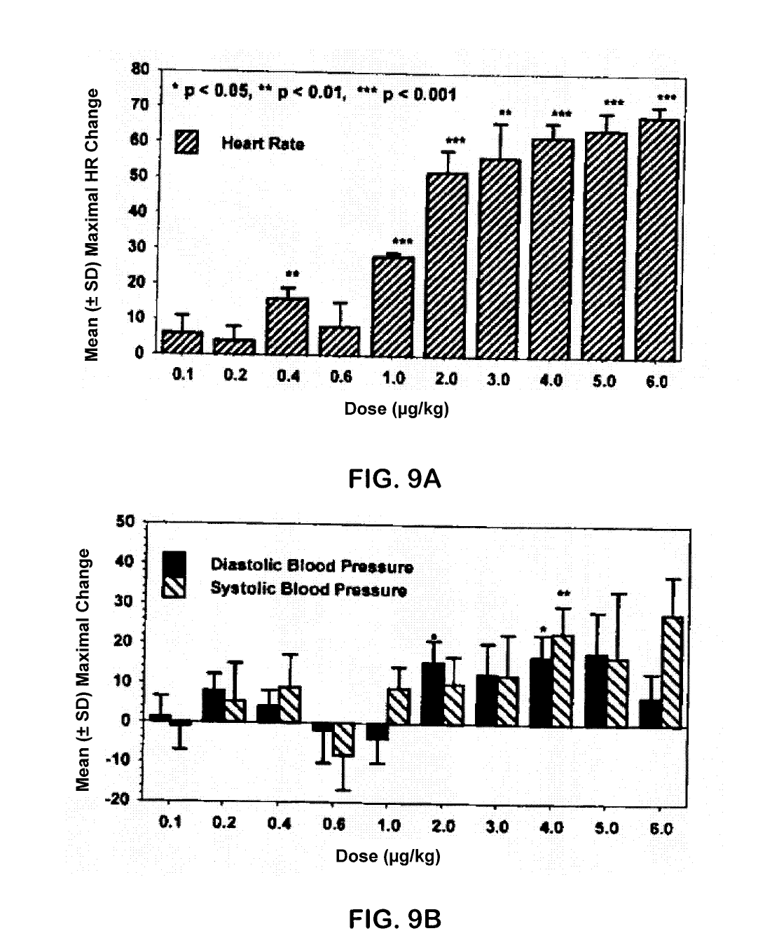

Vital Signs: The maximal effects of binodenoson on blood pressure were variable at doses of 1 μg/kg or lower (FIG. 9B). Mean systolic pressure increased consistently at doses of 1 μg/kg or greater and both mean systolic pressure and mean diastolic pressure were increased at doses of 2 μg/kg or greater. The mean maximal increase in systolic blood pressure from baseline ranged from 8.8 mmHg at the 1 μg/kg dose to 27.9 mmHg at the 6 μg/kg dose. The mean maximal increases in systolic and diastolic blood pressure were statistically significant at the 2- and 4 μg/kg dose levels, respectively (p<0.05).

Binodenoson doses of 0.1 μg/kg or greater were associated with increases in heart rate (FIG. 9A). Maximum increases in heart rate ranged from 29 beats/min at the 1 μg/kg dose to 66.3 beats/min at the 6 μg/kg dose. The changes at doses of 0.4 μg/kg and at doses of 1 μg/kg or greater were statistically significant (p<0.001).

The most prevalent AEs were vasodilation, headache, dizziness, nausea, chest pain, abdominal pain and paresthesia, and were consistent with the pharmacologic properties of the drug. The incidence of AEs was strongly associated with dose and exposure, with a marked increase in the incidence and frequency of the more unpleasant AEs such as chest pain, abdominal pain, dizziness, nausea and vomiting at doses of 4 μg/kg and higher. However, there were no serious AEs and most AEs began during the infusion period, were mild or moderate in severity and resolved within 30 minutes of onset.

In animals, binodenoson produced dose-related hypotension and reflex tachycardia. In this study, peripheral vasodilatory responses were suggested by the occurrence of tingling, flushing, headache, fullness and warmth. Despite the apparent peripheral vasodilation, changes in blood pressure were variable at lower doses, resulting in unchanged mean values and both systolic blood pressure and diastolic blood pressure increased slightly at doses greater than 1 μg/kg. Dose-related increases in heart rate at doses of 1 μg/kg or greater suggest that binodenoson increases heart rate independently of changes in blood pressure and it is uncertain whether the positive chronotropic responses may have obscured drug-induced systemic hypotension. Binodenoson is the first adenosine A_{2A}-receptor-selective agonist to be administered to human beings and this independent increase in heart rate represents a novel finding. Binodenoson did not increase the rate in isolated and denervated atrial preparations and has no affinity for β-adrenergic or muscarinic receptors that might explain such a response. There is preclinical evidence that activation of adenosine A_{2A} receptors enhances norepinephrine release from sympathetic efferent nerve terminals, but such a mechanism has not been defined in human beings. The vasodilator pharmacologic stress agents adenosine and dipyridamole produce modest increases in heart rate and it is likely that this effect enhances their direct coronary vasodilatory responses by increasing myocardial oxygen demand. The same is expected to be true of binodenoson.

The pharmacokinetics of binodenoson were characterized by dose linearity with respect to exposure parameters (Cₘₐₓ and AUC) and rapid disappearance (t_{1/2}=10 minutes) from the systemic circulation after cessation of infusion. Systemic clearance of binodenoson was independent of dose and indicative of rapid removal from the systemic circulation. Although no metabolism studies have been conducted in human beings, in vitro studies with hepatocytes and microsomes suggest low metabolic activity and no significant inhibition of cytochrome P450 enzymes.

The relationship between binodenoson clearance and body weight provides a rational basis for establishing the dose of binodenoson based on body weight to minimize pharmacokinetic variability. In this first-in-human beings study, binodenoson was infused over a period of 10 minutes. The resulting pharmacokinetic data suggested that shorter infusions and bolus doses might provide pharmacodynamic responses consistent with pharmacologic stress imaging.

The simulated binodenoson concentrations after a 1.5 μg/kg dose administered over a period of 30 seconds, 3 minutes and 10 minutes are shown in FIG. 10.

Conclusion: Intravenous binodenoson was well tolerated when administered at doses ranging from 0.4 μg/kg to 6 μg/kg, with pharmacologic effects generally consistent with the pharmacologic properties of A_{2A} receptor activation. Binodenoson pharmacokinetic-pharmacodynamic properties were characterized by dose linearity, short duration of action and rapid removal from the systemic circulation, which are desirable characteristics for this class of drugs.

Although the present invention has been described in terms of specific embodiments, various substitutions of administration methods and conditions can be made as will be known to those skilled in the art. For example, the administration method and/or vehicle may be adjusted to accommodate the imaging technique utilized. Other variations will be apparent to those skilled in the art and are meant to be included herein. The scope of the invention is only to be limited by the claims that follow.

What is claimed is:

1. A method of diagnosing the presence and assessing the severity of coronary artery disease in a human patient having a history of asthma or bronchospasm comprising the steps of:
(a) administering by an intravenous route to the human patient from about 0.1 to about 10 μg/kg of binodenoson to provide coronary artery dilation without simultaneous clinically significant bronchoconstriction as defined by a 20%, or more, decrease in forced expiratory volume in 1 second (FEV₁) from the predrug baseline;
(b) administering an imaging agent to the human patient; and
(c) performing myocardial perfusion imaging on the human patient to detect coronary artery disease.

2. The method of claim 1, wherein binodenoson is administered as a bolus dose to said human patient.

3. The method of claim 2, wherein the bolus dose is about from about 0.5 to about 2.5 μg/kg of binodenoson.

4. The method of claim 2, wherein the bolus dose is from about 0.5 to about 1.5 μg/kg of binodenoson.

5. The method of claim 2, wherein the bolus dose is about 1.5 μg/kg of binodenoson.

6. A method according to claim 5, wherein the imaging agent in step (b) is a radiopharmaceutical.

7. A method according to claim 6, wherein the myocardial perfusion imaging in step (c) comprises scintigraphy.

8. A method of diagnosing the presence and assessing the severity of ventricular dysfunction in a human patient having a history of asthma or bronchospasm comprising the steps of:
(a) administering by an intravenous route to the human patient from about 0.1 to about 10 μg/kg of binodenoson to provide coronary artery dilation without simultaneous clinically significant bronchoconstriction as defined by a 20%, or more, decrease in forced expiratory volume in 1 second (FEV₁) from the predrug baseline; and
(b) performing a ventricular function imaging technique on the human patient to detect ventricular dysfunction.

9. The method of claim 8, wherein binodenoson is administered as a bolus dose to said human patient.

10. The method of claim 9, wherein the bolus dose is from about 0.5 to about 2.5 μg/kg of binodenoson.

11. The method of claim 9, wherein the bolus dose is from about 0.5 to about 1.5 μg/kg of binodenoson.

12. The method of claim 9, wherein the bolus dose is about 1.5 μg/kg of binodenoson.

13. A method according to claim 12, wherein the ventricular function imaging technique in step (b) comprises echocardiography.

14. A method of diagnosing perfusion abnormalities in a human patient having a history of asthma or bronchospasm comprising the steps of:
(a) administering by an intravenous route to the human patient from about 0.1 to about 10 μg/kg of binodenoson to provide coronary artery dilation without simultaneous clinically significant bronchoconstriction as defined by a 20%, or more, decrease in forced expiratory volume in 1 second (FEV₁) from the predrug baseline; and
(b) detecting perfusion abnormalities in the human patient.

15. The method of claim 14, wherein binodenoson is administered as a bolus dose to said human patient.

16. The method of claim 15, wherein the bolus dose is from about 0.5 to about 2.5 μg/kg of binodenoson.

17. The method of claim 15, wherein the bolus dose is from about 0.5 to about 1.5 μg/kg of binodenoson.

18. The method of claim 15, wherein the bolus dose is about 1.5 μg/kg of binodenoson.

19. The method of claim 18, wherein detecting perfusion abnormalities in step (b) comprises measuring coronary blood flow velocity on the human patient to assess the vasodilatory capacity of diseased coronary vessels as compared with disease free coronary vessels.

20. The method of claim 18, wherein detecting perfusion abnormalities in step (b) comprises assessing the vasodilatory capacity (reserve capacity) of diseased coronary vessels as compared with disease free coronary vessels.
